(19) United States
(12) Patent Application Publication
(10) Pub. No.: US 2025/0036023 A1
(43) Pub. Date: Jan. 30, 2025

(54) LITHOGRAPHY COMPOSITIONS AND METHODS FOR FORMING RESIST PATTERNS AND/OR MAKING SEMICONDUCTOR DEVICES

(71) Applicant: The Research Foundation for the State University of New York, Albany, NY (US)

(72) Inventors: Munsaf Ali, Albany, NY (US); Robert Brainard, Albany, NY (US)

(21) Appl. No.: 18/710,234

(22) PCT Filed: Nov. 15, 2022
(86) PCT No.: PCT/US2022/050003

§ 371 Date: May 15, 2024

Related U.S. Application Data
(60) Provisional application No. 63/279,402, filed on Nov. 15, 2021.

(51) Int. Cl. G03F 7/004; C07F 7/22; C07F 9/92; C07F 11/00; G03F 7/16; H01L 21/027

(52) U.S. Cl. CPC G03F 7/0042; C07F 7/2224; C07F 9/92; C07F 11/00; G03F 7/162; G03F 7/168; H01L 21/0274

(57) ABSTRACT

The present disclosure relates to compounds and use thereof as lithographic compositions such as EUV photoresist films. More particularly, embodiments of the disclosure provide lithography compositions and methods of depositing radiation sensitive films, which can be used for patterning applications with UV light, EUV light or electron-beam radiation to form high resolution patterns with low line width roughness. In embodiments, novel ligands are provided for forming radiation sensitive film compositions.

### FIELD OF THE INVENTION

The present disclosure is generally in the fields of organic chemistry and electronic device manufacturing. More particularly, embodiments of the disclosure provide lithography compositions and methods of depositing radiation sensitive films, which can be used for patterning applications with UV light, EUV light or electron-beam radiation to form high resolution patterns with low line width roughness.

### BACKGROUND

In forming electronic devices such as semiconductor-based structures, materials are patterned to integrate a predetermined structure. Structures are typically formed through sequential deposition and etching steps through which a pattern is formed of the various materials. In this way, many devices such as transistors can be formed in a high-density area.

Organic compositions can be used as radiation patterned resists so that a radiation pattern alters the chemical structure of the organic compositions corresponding with the pattern. For example, processes for the patterning of semiconductor wafers can include lithographic transfer of a desired image from a thin film of organic radiation-sensitive material. The patterning of the resist generally involves several process sequences including exposing the resist to an energy source, such as through a mask, to record a latent image and then developing and removing selected regions of the resist. For a positive-tone resist, the exposed regions are altered to make such regions selectively removable, while for a negative-tone resist, the unexposed regions are selectively removable.

Generally, the pattern is developed with a liquid solution to remove a selectively sensitive portion of the resist while the other portions of the resist act as a protective etch-resistant layer. The substrate can be selectively etched through holes in the remaining areas of the protective resist layer. Alternatively, materials can be deposited into the exposed regions of the underlying substrate through the developed holes in the remaining areas of the protective resist layer. Ultimately, the protective resist layer is removed. Generally, the process can be repeated to form additional layers of patterned material. Additional processing sequences can be used, such as the deposition of conductive materials or implantation of dopants. In the fields of micro- and nanofabrication, feature sizes in integrated circuits have become very small to achieve high-integration densities and improve circuit function.

Prior art-of-interest includes U.S. Pat. No. 10,228,618 entitled Organotin oxide hydroxide patterning compositions, precursors, and patterning (herein incorporated entirely by reference), however, the methods do not provide high resolution lithography patterning coatings based on the chemistry, compositions, and/or methods of the present disclosure.

Prior art-of-interest also includes U.S. Pat. No. 11,156,920 entitled Lithography composition, a method for forming resist patterns and a method for making semiconductor devices (herein incorporated entirely by reference), however, the disclosure does not provide the compositions of the present disclosure.

As microelectronic device sizes become smaller, challenges exist with current resist films. New photolithographic films are needed for a variety of applications in microelectronic devices. Accordingly, there is a continuing need in the art for new lithography compositions, methods for forming resist patterns using one or more lithography compositions, and semiconductor device manufacturing methods using the lithography compositions.

### SUMMARY

In embodiments, the present disclosure provides new lithography compositions, methods for forming resist patterns using a lithography composition, and semiconductor device manufacturing methods using the lithography compositions in a photolithography method of the present disclosure.

In embodiments, the present disclosure provides a lithography composition represented by the below formula,

[MₐN_{b}O_{c}H_{d}BₑR_{f}X_{g}(WCOR′)ₕ(OR″)ᵢ(WCOCOY)ⱼ(COY)ₖ]^{l},
wherein: R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH₂C₆H₄F, —CH₂C₆H₃F₂, —CH₂C₆H₂F₃, —CH₂C₆F₅, —CH(CH₃)C₆H₅, —CH(CH₃)C₁₀H₇, —CH(CH₃)C₆H₄C₆H₅, or —CH(CH₃)C₆H₄C(CH₃)₃;
R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, or —C₆H₄Cl;
R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH₂CH═CH₂, —CH₂C≡CH, —CH₂C≡N, —CH₂C₆H₅, —C₆H₄CH═CH₂, —CH(CH₃)C₆H₅, or —C₆H₄C(CH₃)═CH₂;
M is each independently an element selected from the group of antimony (Sb), tellurium (Te), tin (Sn) or iodine (I); N, when present, is each independently an element selected from the group consisting of tin (Sn), antimony (Sb), tellurium (Te), bismuth (Bi) iodine (I) and indium (In); H, when present, is each independently hydrogen; O is each independently oxygen; B, when present, is each independently selected from W, C₂O₄, SO₄, PO₄, (CH₂)₂C₆H₄(CH₂)₂, —OO—, —OCH₂O—, —OCH₂CH₂O—, —OCH₂CH₂CH₂O—, —OCH₂CH₂CH₂CH₂O—, —OC₆H₄O—, —OCH₂C₆H₄CH₂O—, —OCH₂CH═CHCH₂O—, —OCH₂C≡CCH₂O—, —SCH₂CH₂S—, —SCH₂CH₂CH₂S—, —SCH₂CH₂CH₂CH₂S—, —SC₆H₄S—, —SCH₂C₆H₄CH₂S—, —SCH₂CH═CHCH₂S—, —SCH₂C≡CCH₂S—, —O₂CCH₂CO₂—, —O₂CCH₂CH₂CO₂—, —O₂CCH₂C₆H₄CH₂CO₂—, —NHC═ONH—, —CH₂—, —CH₂CH₂—, —CH₂CH₂CH₂—, —CH(CH₃)—, —CH₂CH═CHCH₂—, —CH₂C≡CCH₂—, or —CH₂C₆H₄CH₂—; X, when present, is each independently selected from F, Cl, Br, CN, or C₂O₄; W, when present, is each independently selected from the group consisting of —CH₂, NR′, S, and O; and Y when present, is each independently selected from the group consisting of —WR′, and —R′, wherein a=1-8; b=0-5; c=0-20; d=0-20; e=0-10; f=0-20; g=0-20; h=0-5; i=0-5; j=0-5; k=0-6; and l=charge on an ion or a complex selected from: −4, −3, −2, −1, 0, +1, +2, +3, +4. In embodiments, at least two or more, at least three or more, or at least four or more of M, N, O, H, B, R, X, WCOR′, OR″, WCOCOY, or COY are present. In embodiments, one of WCOR′, WCOCOY, or COY is present.

In embodiments, the present disclosure includes a lithography composition including: an organic solvent, and a composition represented by the below formula,

[MₐN_{b}O_{c}H_{d}BₑR_{f}X_{g}(WCOR′)ₕ(OR″)ᵢ(WCOCOY)ⱼ(COY)ₖ]^{l},
wherein: R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH₂C₆H₄F, —CH₂C₆H₃F₂, —CH₂C₆H₂F₃, —CH₂C₆F₅, —CH(CH₃)C₆H₅, —CH(CH₃)C₁₀H₇, —CH(CH₃)C₆H₄C₆H₅, or —CH(CH₃)C₆H₄C(CH₃)₃;
R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, or —C₆H₄Cl;
R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH₂CH═CH₂, —CH₂C≡CH, —CH₂C≡N, —CH₂C₆H₅, —C₆H₄CH═CH₂, —CH(CH₃)C₆H₅, or —C₆H₄C(CH₃)═CH₂;
M is each independently an element selected from the group of antimony (Sb), tellurium (Te), tin (Sn) or iodine (I); N, when present, is each independently an element selected from the group consisting of tin (Sn), antimony (Sb), tellurium (Te), bismuth (Bi) iodine (I) and indium (In); H, when present, is each independently hydrogen; O is each independently oxygen; B, when present, is each independently selected from W, C₂O₄, SO₄, PO₄, (CH₂)₂C₆H₄(CH₂)₂, —OO—, —OCH₂O—, —OCH₂CH₂O—, —OCH₂CH₂CH₂O—, —OCH₂CH₂CH₂CH₂O—, —OC₆H₄O—, —OCH₂C₆H₄CH₂O—, —OCH₂CH═CHCH₂O—, —OCH₂C≡CCH₂O—, —SCH₂CH₂S—, —SCH₂CH₂CH₂S—, —SCH₂CH₂CH₂CH₂S—, —SC₆H₄S—, —SCH₂C₆H₄CH₂S—, —SCH₂CH═CHCH₂S—, —SCH₂C≡CCH₂S—, —O₂CCH₂CO₂—, —O₂CCH₂CH₂CO₂—, —O₂CCH₂C₆H₄CH₂CO₂—, —NHC═ONH—, —CH₂—, —CH₂CH₂—, —CH₂CH₂CH₂—, —CH(CH₃)—, —CH₂CH═CHCH₂—, —CH₂C≡CCH₂—, or —CH₂C₆H₄CH₂—; X, when present, is each independently selected from F, Cl, Br, CN, or C₂O₄; W, when present, is each independently selected from the group consisting of —CH₂, NR′, S, and O; and Y when present, is each independently selected from the group consisting of —WR′, and —R′, wherein a=1-8; b=0-5; c=0-20; d=0-20; e=0-10; f=0-20; g=0-20; h=0-5; i=0-5; j=0-5; k=0-6; and l=charge on an ion or a complex selected from: −4, −3, −2, −1, 0, +1, +2, +3, +4. In embodiments, at least two or more, at least three or more, or at least four or more of M, N, O, H, B, R, X, WCOR′, OR″, WCOCOY, or COY are present. In embodiments, one of WCOR′, WCOCOY, or COY is present.

In embodiments, the present disclosure relates to a method for forming a resist pattern using a lithography composition, including: contacting a substrate with a lithography composition of the present disclosure to form a thin radiation sensitive film of the lithography composition atop the substrate.

In embodiments, the present disclosure relates to a method for forming a semiconductor device using one or more lithography compositions of the present disclosure in a photolithography method of the present disclosure. In embodiments, a method for forming a semiconductor device includes contacting a substrate with a lithography composition of the present disclosure to form a thin radiation sensitive film of the lithography composition atop the substrate.

In embodiments, the present disclosure relates to a method for forming an electronic device using the lithography compositions in a photolithography method of the present disclosure. In embodiments, a method for forming an electronic device includes contacting a substrate with a lithography composition of the present disclosure to form a thin radiation sensitive film of the lithography composition atop the substrate.

In embodiments, the present disclosure relates to a substrate and a radiation sensitive coating including a composition of the present disclosure.

In embodiments, the present disclosure related to a substrate including an inorganic semiconductor layer and a radiation sensitive coating material atop a surface, wherein the radiation sensitive coating includes a lithographic composition of the present disclosure. In some embodiments, the radiation coating material or film of the present disclosure can be patterned with EUV light at a wavelength of 13.5 nm in a pattern of 16-nm lines, with a line width roughness of no more than about 4 nm. In embodiments, the radiation sensitive coating material can include metal, such as Te, and can include at least 5 weight percent metal and in other embodiments at least about 20 weight percent metal. In embodiments, the radiation sensitive coating is characterized as amorphous.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 depicts a flow diagram illustrating a process 100 for forming an electronic device such as a semiconductor device using the lithography compositions in a photolithography method of the present disclosure.

FIGS. 2A, 2B, and 2C show a schematic diagram of an electronic device such as a semiconductor device, according to embodiments of the present disclosure.

FIG. 3 presents the results of contrast curves for MA-251.

FIG. 4 presents the results of contrast curves for of RB-54.

FIG. 5 presents the results of contrast curves of RB-124.

FIG. 6 presents the results of contrast curves of NU-87.

FIG. 7 displays SEM imaging of NU-87.

FIG. 8 presents the results of contrast curves of RB-56.

FIG. 9. presents the results of contrast curves of RB-129.

FIG. 10 displays SEM imaging of RB-129.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The presently disclosed subject matter will now be described more fully and representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Throughout the specification and claims, a given chemical formula or name shall encompass all optical and stereoisomers, as well as racemic mixtures where such isomers and mixtures exist.

In embodiments, the present disclosure provides new lithography compositions, methods for forming of resist patterns using a lithography composition, and semiconductor device manufacturing methods using the lithography compositions in a photolithography method of the present disclosure.

In embodiments, the present disclosure provides a composition, such as a lithographic composition, represented by the below formula,

[MₐN_{b}O_{c}H_{d}BₑR_{f}X_{g}(WCOR′)ₕ(OR″)ᵢ(WCOCOY)ⱼ(COY)ₖ]^{l},
wherein: R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH₂C₆H₄F, —CH₂C₆H₃F₂, —CH₂C₆H₂F₃, —CH₂C₆F₅, —CH(CH₃)C₆H₅, —CH(CH₃)C₁₀H₇, —CH(CH₃)C₆H₄C₆H₅, or —CH(CH₃)C₆H₄C(CH₃)₃;
R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, or —C₆H₄Cl;
R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH₂CH═CH₂, —CH₂C≡CH, —CH₂C≡N, —CH₂C₆H₅, —C₆H₄CH═CH₂, —CH(CH₃)C₆H₅, or —C₆H₄C(CH₃)=CH₂;
M is each independently an element selected from the group of antimony (Sb), tellurium (Te), tin (Sn) or iodine (I);
N, when present, is each independently an element selected from the group consisting of indium (In), tin (Sn), antimony (Sb), tellurium (Te), bismuth (Bi) and iodine (I);
H, when present, is each independently hydrogen;
O is each independently oxygen;
B, when present, is each independently selected from W, C₂O₄, SO₄, PO₄, or (CH₂)₂C₆H₄(CH₂)₂;
X, when present, is each independently selected from F, Cl, Br, CN, or C₂O₄,
W, when present, is each independently an element or a compound selected from the group consisting of —CH₂, NR′, S, and O;
Y when present, is each independently an element or compound selected from the group consisting of —WR′, and —R′, wherein
a=1-8; b=0-5; c=0-20; d=0-20; e=0-10; f=0-20; g=0-20; h=0-5; i=0-5; j=0-5; k=0-6; and l=charge on an ion or a complex selected from: −4, −3, −2, −1, 0, +1, +2, +3, +4.

In embodiments, the lithography compositions are suitable for forming radiation sensitive films such as photosensitive resists which, when exposed to light such as EUV, becomes more or less soluble in developer relative to the unexposed portions of the resist. Such materials are used in making semiconductor devices such as microcircuits. Advantages of the compositions of the present disclosure include, inter alia, positive-tone response for palladium oxalates, extremely low line-edge roughness for tin carboxylates, and extraordinary photo speed for antimony carboxylates.

In embodiments, WCOR′ is selected from the group consisting of CH₂CO₂C(CH₃)₃, O₂CC(CH₃)=CH₂, O₂CCH═CH₂, O₂CC(CH₃)₃, CH₂CO₂C(CH₃)₃, C₂O₄, O₂CCH₂CH₂Cl, O₂CC₆H₄CH═CH₂, and combinations thereof.

In embodiments, WCOCOY and is selected from the group consisting of COCO₂CH₂CH₃, CH₂COCO₂CH₂CH₃, COCO₂C(CH₃)₃, and combinations thereof.

In embodiments, COY and is selected from the group consisting of C(O)C₆H₅, C(O)C₆H₄OCH₃, C(O)C₆H₅, and combinations thereof.

In embodiments, B is selected from the group consisting of W, C₂O₄, SO₄, PO₄, (CH₂)₂C₆H₄(CH₂)₂, —OO—, —OCH₂O—, —OCH₂CH₂O—, —OCH₂CH₂CH₂O—, —OCH₂CH₂CH₂CH₂O—, —OC₆H₄O—, —OCH₂C₆H₄CH₂O—, —OCH₂CH═CHCH₂O—, —OCH₂C≡CCH₂O—, —SCH₂CH₂S—, —SCH₂CH₂CH₂S—, —SCH₂CH₂CH₂CH₂S—, —SC₆H₄S—, —SCH₂C₆H₄CH₂S—, —SCH₂CH═CHCH₂S—, —SCH₂C≡CCH₂S—, —O₂CCH₂CO₂—, —O₂CCH₂CH₂CO₂—, —O₂CCH₂C₆H₄CH₂CO₂—, —NHC═ONH—, —CH₂—, —CH₂CH₂—, —CH₂CH₂CH₂, —CH(CH₃)—, —CH₂CH═CHCH₂—, —CH₂C≡CCH₂—, or —CH₂C₆H₄CH₂—; and combinations thereof.

In embodiments, the compositions are suitable for use as EUV photoresist films, deposited atop a substrate in an electronic device manufacturing process, or semiconductor manufacturing process flow. In embodiments, it is understood that if a value in the formula is zero (0), then the constituent is not present in the composition.

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein M=I, a=1, N=Sn, Te, Sb, b=2, c=6, d=1, B=0, e=0, R=R; f=0 or 4 when N=Sn or Te, and f=0 or 6 when N=Sb; X=0, g=0, WCOR′=0, h=0, R″=0, i=0; WCOCOY=COCO₂C(CH₃)₃, j=0 or 4 when N=Sn or Te, and j=0 or 6 when N=Sb; COY=COC₆H₅ or COC₆H₄OCH₃, k=0 or 4 when N=Sn or Te, and k=0 or 6 when N=Sb; l=0.

For example, compositions are represented by the below multinuclear cluster structure Type 1 formulas:

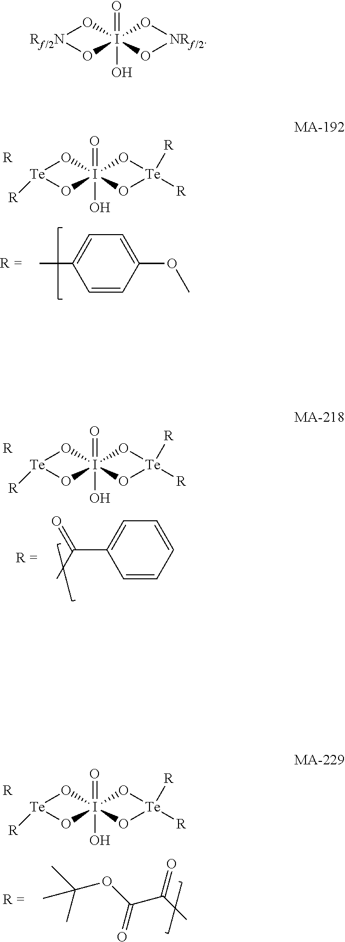

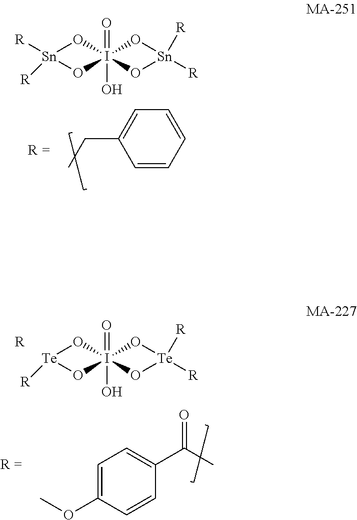

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein: M=Sb, Sn, Te; a=2, N=0, b=0, c=1, d=0, B=0, e=0, R=R; f=4 when N=Sn or Te, and f=6 when N=Sb; X=0, g=0, W=—O—, R′=R′, h=2, R″=0, i=0, COCOOY=0, j=0, COY=0, k=0, l=0. For example, compositions are represented by the below multinuclear cluster structure Type 2 formulas:

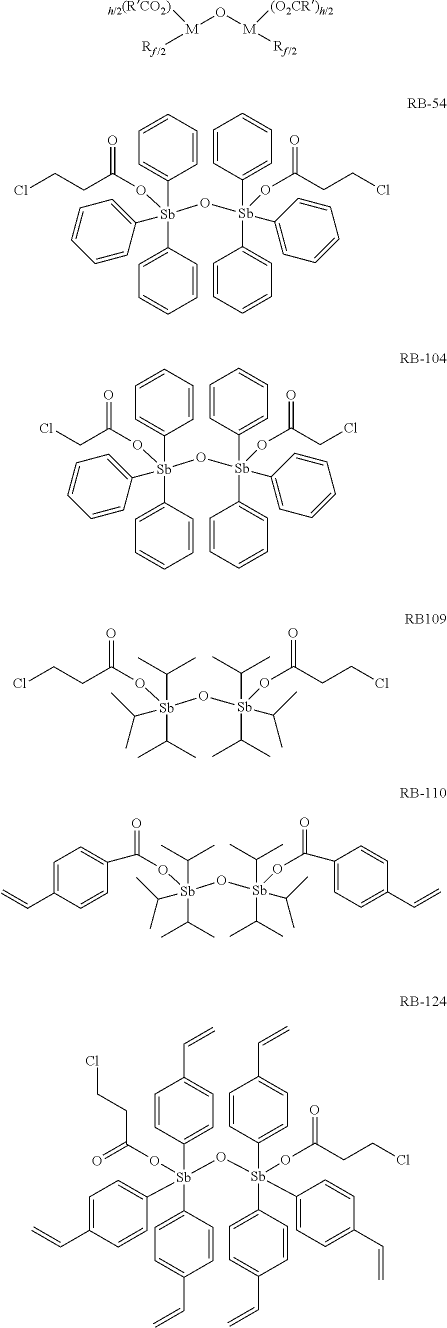

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein: M=Sb, Sn, Te; a=2, N=0, b=0, c=0, d=0, e=0, R=R; f=4 when N=Sn or Te, and f=6 when N=Sb; X=0, g=0, W=—O—, R′=R′, h=2, R″=0, i=0, COCOOY=0, j=0, COY=0, k=0, l=0 and B=—OO—, —OCH₂O—, —OCH₂CH₂O—, —OCH₂CH₂CH₂O—, —OCH₂CH₂CH₂CH₂O—, —OC₆H₄O—, —OCH₂C₆H₄CH₂O—, —OCH₂CH═CHCH₂O—, —OCH₂C≡CCH₂O—, —SCH₂CH₂S—, —SCH₂CH₂CH₂S—, —SCH₂CH₂CH₂CH₂S—, —SC₆H₄S—, —SCH₂C₆H₄CH₂S—, —SCH₂CH═CHCH₂S—, —SCH₂C≡CCH₂S—, —O₂CCH₂CO₂—, —O₂CCH₂CH₂CO₂—, or —NHC═ONH—. For example, compositions are represented by the below multinuclear cluster structure Type 3 formulas: (Type 3 is represented by a prophetic formula and is not presented in Table 1)

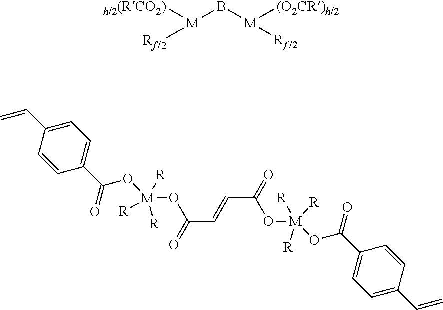

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein: M=Te, a=1, N=Sn or Sb, b=3, c=6, d=0, B=0, e=0, R=R; f=6 when N=Sn, and f=9 when N=Sb; X=0, g=0, WCOR′=0, h=0, R″=0, i=0, COCOOY=0, j=0, COY=0, k=0, l=0. For example, compositions are represented by the below multinuclear cluster structure Type 4 formulas:

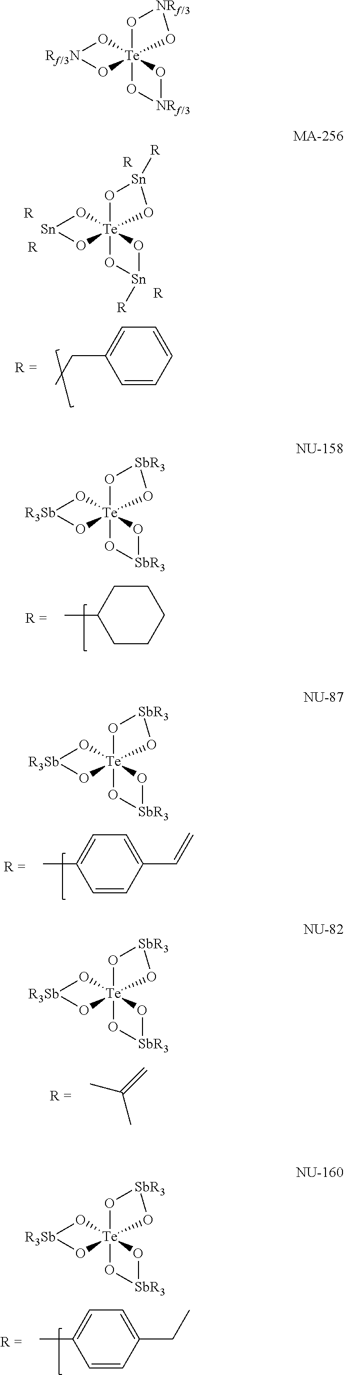

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein M=Te, a=4, N=0, b=0, c=6, d=0, B=0, e=0, R=R, f=0 or 6, X=0, g=0, WCOR′=0, h=0, R″=0, i=0, WCOCOY=0, j=0, l=0; COY=COC₆H₅, k=0 or 6. For example, compositions are represented by the below Multinuclear Cluster Type 5 formulas:

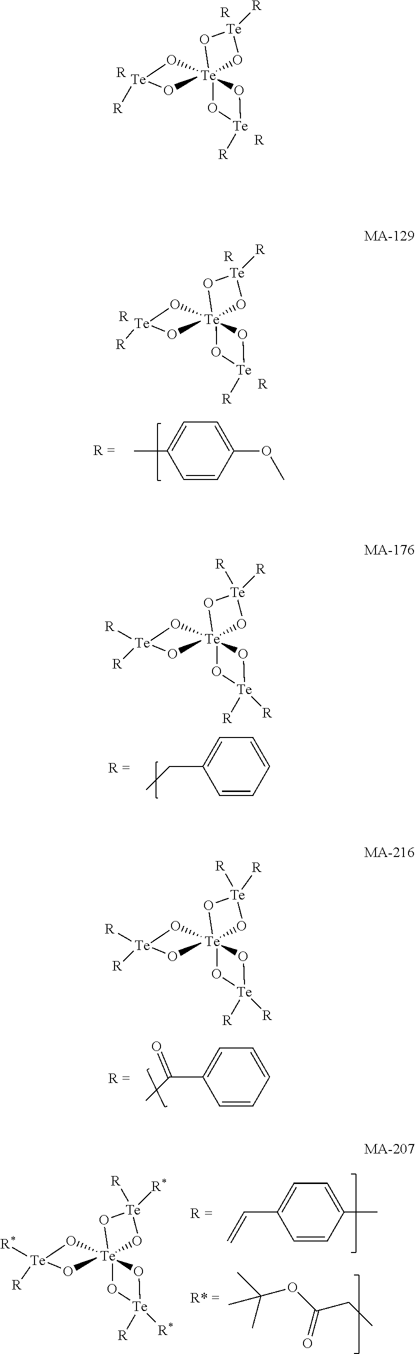

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein: M=Sb or Sn, a=2, N=0, b=0, c=0, d=0, B=CH₂, e=1, R=R, f=4, X=0, g=0, W=—O—, R′=R′; h=2 if M=Sn, or h=4 if M=Sb; R″=0, i=0, COCOOY=0, j=0, COY=0, k=0, l=0. For example, compositions are represented by the below multinuclear cluster structure Type 6 formulas:

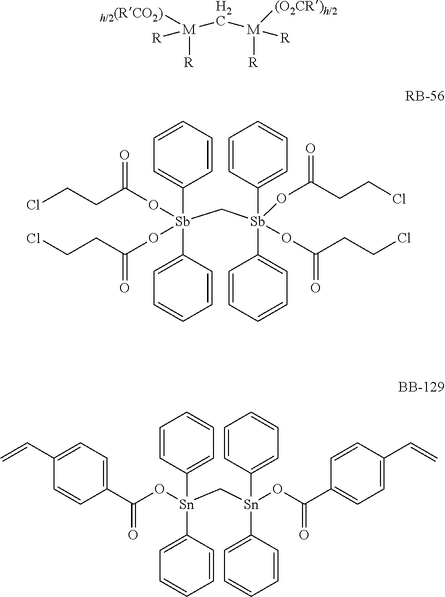

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein: M=Sb or Sn, a=2, N=0, b=0, c=0, d=0; B=—CH₂CH₂—, —CH₂CH₂CH₂—, —CH(CH₃)—, —CH₂CH═CHCH₂—, —CH₂C≡CCH₂—, or —CH₂C₆H₄CH₂—; e=1, R=R, f=4, X=0, g=0, W=—O—, R′=R′; h=2 if M=Sn, or h=4 if M=Sb; R″=0, i=0, COCOOY=0, j=0, COY=0, k=0, l=0. For example, compositions are represented by the below multinuclear cluster structure Type 7 formulas: (Type 7 is represented by a prophetic formula and is not presented in Table 1)

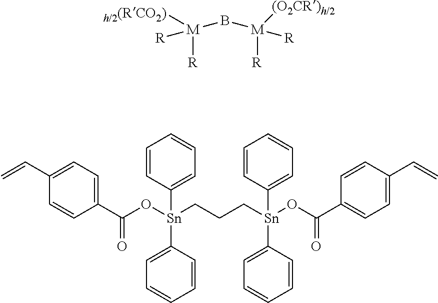

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein M=Sb, Te or Sn, a=2, N=0, b=0, c=0, d=0, B=—O₂CCO₂— or —O₂SO₂—, e=1, R=R, f=0 or 4 if M=Te or Sn, f=0 or 6 if M=Sb; X=X, g=0 or 2; WCOR′=CH₂CO₂C(CH₃)₃, h=0 or 4 if M=Te or Sn, h=0 or 6 if M=Sb; OR″=OCH₃ or OC(CH₃)₃, i=0 or 2, WCOCOY=CH₂COCO₂CH₂CH₃ or COCO₂CH₂CH₃, j=0 or 4 if M=Te or Sn, j=0 or 6 if M=Sb; COY=COC₆H₄Cl, k=0 or 4 if M=Te or Sn, k=0 or 6 if M=Sb; l=0. For example, compositions are represented by the below Multinuclear Cluster Type 8 formulas:

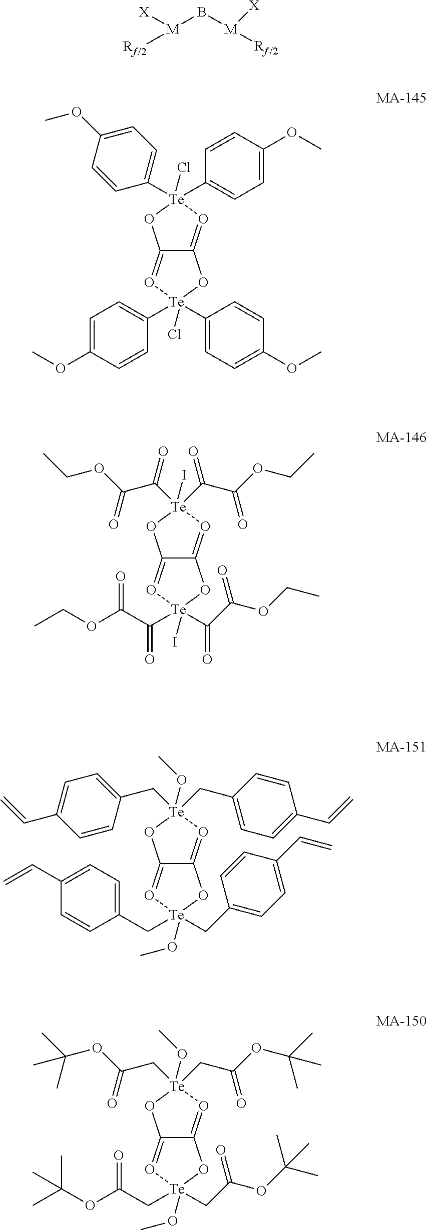

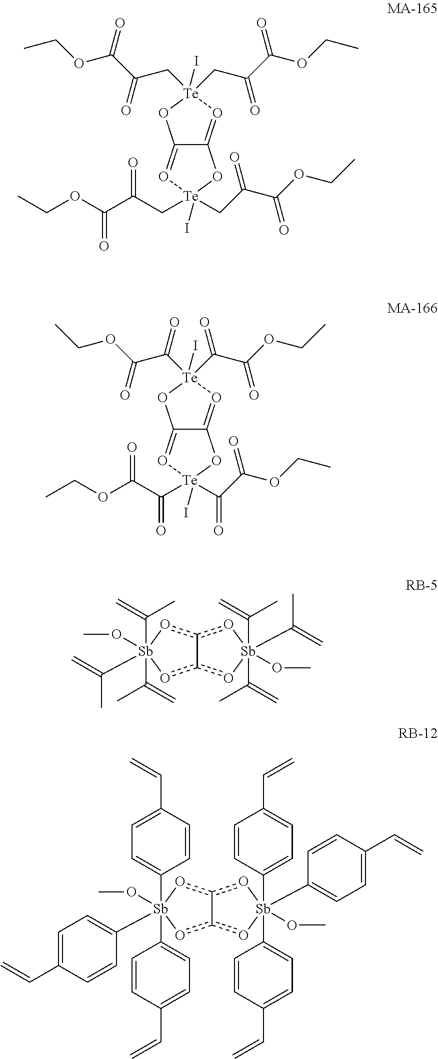

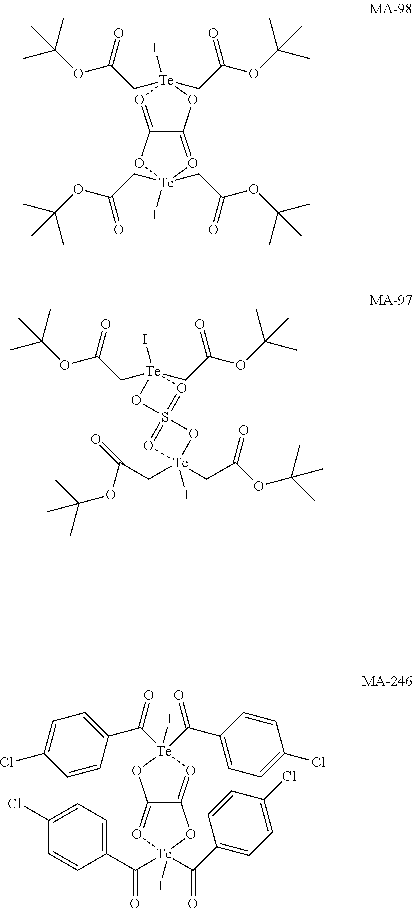

In one embodiment, the present disclosure includes a lithographic composition represented by the formula, [MₐN_{b}O_{c}H_{d}BₑR_{f}X_{g}(WCOR′)ₕ(OR″)ᵢ(WCOCOY)ⱼ(COY)ₖ]^{l}, wherein: M=Te, a=1, N=Sn, Te or Sb, b=2, c=6, d=2, B=0, e=0, R=R; f=4 if N=Sn or Te or f=6 if N=Sb; X=0, g=0, WCOR′=0, h=0, R″=0, i=0, COCOOY=0, j=0, COY=0, k=0, l=0. For example, compositions are represented by the below multinuclear cluster structure Type 9 formulas: (Type 9 is represented by a prophetic formula and is not presented in Table 1)

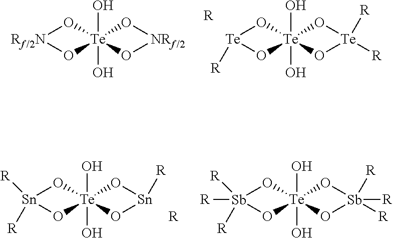

In embodiments, the compositions and lithographic compositions of the present disclosure may possess one or more stereocenters, and each stereocenter may exist independently in either the R or S configuration.

It is also to be understood that the compositions and/or lithographic compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

In embodiments, the present disclosure includes a lithographic composition represented by the below formula,

[MₐN_{b}O_{c}H_{d}BₑR_{f}X_{g}(WCOR′)ₕ(OR″)ᵢ(WCOCOY)ⱼ(COY)ₖ]^{l},

wherein:
R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH₂C₆H₄F, —CH₂C₆H₃F₂, —CH₂C₆H₂F₃, —CH₂C₆F₅, —CH(CH₃)C₆H₅, —CH(CH₃)C₁₀H₇, —CH(CH₃)C₆H₄C₆H₅, or —CH(CH₃)C₆H₄C(CH₃)₃;
R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, or —C₆H₄Cl;
R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH₂CH═CH₂, —CH₂C≡CH, —CH₂C≡N, —CH₂C₆H₅, —C₆H₄CH═CH₂, —CH(CH₃)C₆H₅ or —C₆H₄C(CH₃)=CH₂;
M is each independently an element selected from the group of antimony (Sb), tellurium (Te), tin (Sn) or iodine (I);
N, when present, is each independently an element selected from the group consisting of indium (In), tin (Sn), antimony (Sb), tellurium (Te), bismuth (Bi) and iodine (I);
H, when present, is each independently hydrogen;
O is each independently oxygen;
B, when present, is each independently selected from W, C₂O₄, SO₄, PO₄, or (CH₂)₂C₆H₄(CH₂)₂;
X, when present, is each independently selected from F, Cl, Br, CN, or C₂O₄;
W, when present, is each independently an element or a compound selected from the group consisting of —CH₂, NR′, S, and O;
Y when present, is each independently an element or compound selected from the group consisting of —WR′, and —R′, wherein
a=1-8, 1-5, or 1, 2, 3, 4, 5, 6, 7, or 8; b=0-5, 2-6, or 1, 2, 3, 4, 5, or 6; c=0-20, 1-10, or 2, 3, 4, 5, 6, 7 or 8; d=0-20, 1-2, 1, or 2; e=0-10, 1-5, 1, or 2; f=0-20, 0.5-16, 2, 3, 4, 5, 6, 7, 8, 9, 10, 11, 12, 13, 14, 15, or 16; g=0-20, 1-4, 1, 2, 3, or 4; h=0-5, 1-4, 1, 2, 3, or 4; i=0-5, 1-2, 1, or 2; j=0-5. 1-4, 1, 2, 3, or 4; k=0-6, 1-6, 1, 2, 3, 4, 5, or 6; and l=charge on an ion or a complex selected from: −4, −3, −2, −1, 0, +1, +2, +3, +4.

In embodiments, M, a, and B are present. In embodiments, M, a, N, and b are present. In embodiments, M, a, and R are present. In embodiments, M, a, and WCOR′ are present. In embodiments, M, a, R and WCOR′ are present. In embodiments, M, a, B and WCOR′ are present. In embodiments, M, a, B and R are present. In embodiments, at least two or more of M, N, O, H, B, R, X, WCOR′, OR″, WCOCOY, or COY are present. In embodiments, at least three or more of M, N, O, H, B, R, X, WCOR′, OR″, WCOCOY, or COY are present. In embodiments, at least four or more of M, N, O, H, B, R, X, WCOR′, OR″, WCOCOY, or COY are present. In embodiments, N is an element selected from the group consisting of indium, tin, antimony, and bismuth. In embodiments, R is selected from the group consisting of —CH═CH₂, —C(CH₃)=CH₂, —CH₂CH═CH₂, —CH₂C≡CH, —CH₂C≡N. In embodiments, a=1-2; and b=1-2. In embodiments, R″ is selected from both —H and the group —CH₃, —CH₂CH₃, —CH(CH₃)₂, —C(CH₃)₃, —C₆H₅, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂. In embodiments, h=charge on an ion or a complex selected from: −2, −1, 0, +1, +2. In embodiments, M is tellurium. In embodiments, M is iodine. In embodiments, R″ is selected from both —H and the group —CH₃, —CH₂CH₃, —CH(CH₃)₂, —C(CH₃)₃, —C₆H₅, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂. In embodiments, R is —C(CH₃)=CH₂. In embodiments, N is an element selected from the group of tellurium or iodine. In embodiments, M is tellurium; and N is tellurium. In embodiments, M is iodine; and N is iodine. In embodiments, R is selected from the group consisting of —CH═CH₂, —C(CH₃)=CH₂, —CH₂CH═CH₂, —CH₂C≡CH, —CH₂C≡N. In embodiments, R is —C(CH₃)=CH₂. In embodiments, a=1-2; and b=1-3. In embodiments, the composition is characterized as an EUV photoresist.

In embodiments, WCOR′ is selected from the group consisting of CH₂CO₂C(CH₃)₃, O₂CC(CH₃)=CH₂, O₂CCH═CH₂, O₂CC(CH₃)₃, CH₂CO₂C(CH₃)₃, C₂O₄, O₂CCH₂CH₂Cl, O₂CC₆H₄CH═CH₂, and combinations thereof.

In embodiments, WCOCOY and is selected from the group consisting of COCO₂CH₂CH₃, CH₂COCO₂CH₂CH₃, COCO₂C(CH₃)₃, and combinations thereof.

In embodiments, COY and is selected from the group consisting of C(O)C₆H₅, C(O)C₆H₄OCH₃, C(O)C₆H₅, and combinations thereof.

In embodiments, B is selected from the group consisting of C₂O₄, SO₄, (CH₂)₂C₆H₄(CH₂)₂, PO₄, CH₂, and combinations thereof.

In embodiments, the present disclosure includes a coating solution including: an organic solvent, and an organometallic composition represented by the formula

[MₐN_{b}O_{c}H_{d}BₑR_{f}X_{g}(WCOR′)ₕ(OR″)ᵢ(WCOCOY)ⱼ(COY)ₖ]^{l},

wherein:
R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH₂C₆H₄F, —CH₂C₆H₃F₂, —CH₂C₆H₂F₃, —CH₂C₆F₅, —CH(CH₃)C₆H₅, —CH(CH₃)C₁₀H₇, —CH(CH₃)C₆H₄C₆H₅, or —CH(CH₃)C₆H₄C(CH₃)₃;
R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, or —C₆H₄Cl;
R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH₂CH═CH₂, —CH₂C≡CH, —CH₂C≡N, —CH₂C₆H₅, —C₆H₄CH═CH₂, —CH(CH₃)C₆H₅ or —C₆H₄C(CH₃)=CH₂;
M is each independently an element selected from the group of antimony (Sb), tellurium (Te), tin (Sn) or iodine (I);
N, when present, is each independently an element selected from the group consisting of indium (In), tin (Sn), antimony (Sb), tellurium (Te), bismuth (Bi) and iodine (I);
H, when present, is each independently hydrogen;
O is each independently oxygen;
B, when present, is each independently selected from W, C₂O₄, SO₄, PO₄, or (CH₂)₂C₆H₄(CH₂)₂;
X, when present, is each independently selected from F, Cl, Br, CN, or C₂O₄;
W, when present, is each independently an element or a compound selected from the group consisting of —CH₂, NR′, S, and O;
Y when present, is each independently an element or compound selected from the group consisting of —WR′, and —R′, wherein
a=1-8, 1-5, or 1, 2, 3, 4, 5, 6, 7, or 8; b=0-5, 2-6, or 1, 2, 3, 4, 5, or 6; c=0-20, 1-10, or 2, 3, 4, 5, 6, 7 or 8; d=0-20, 1-2, 1, or 2; e=0-10, 1-5, 1, or 2; f=0-20, 0.5-16, 2, 3, 4, 5, 6, 7, 8, 9, 10, 11, 12, 13, 14, 15, or 16; g=0-20, 1-4, 1, 2, 3, or 4; h=0-5, 1-4, 1, 2, 3, or 4; i=0-5, 1-2, 1, or 2; j=0-5, 1-4, 1, 2, 3, or 4; k=0-6, 1-6, 1, 2, 3, 4, 5, or 6; and l=charge on an ion or a complex selected from: −4, −3, −2, −1, 0, +1, +2, +3, +4, or a mixture thereof. In embodiments, the solvent is an alcohol, an ester, or a mixture thereof.

In embodiments, WCOR′ is selected from the group consisting of CH₂CO₂C(CH₃)₃, O₂CC(CH₃)=CH₂, O₂CCH═CH₂, O₂CC(CH₃)₃, CH₂CO₂C(CH₃)₃, C₂O₄, O₂CCH₂CH₂Cl, O₂CC₆H₄CH═CH₂, and combinations thereof.

In embodiments, WCOCOY and is selected from the group consisting of COCO₂CH₂CH₃, CH₂COCO₂CH₂CH₃, COCO₂C(CH₃)₃, and combinations thereof.

In embodiments, COY and is selected from the group consisting of C(O)C₆H₅, C(O)C₆H₄OCH₃, C(O)C₆H₅, and combinations thereof. In embodiments, B is selected from the group consisting of C₂O₄, SO₄, (CH₂)₂C₆H₄(CH₂)₂, PO₄, CH₂, and combinations thereof.

In embodiments, the present disclosure includes a method for forming a radiation patternable coating, the method including: contacting a coating solution of the present disclosure with a substrate under conditions suitable for forming a film atop the substrate. In embodiments, the substrate is heated to a temperature from about 30 degrees Celsius to about 250 degrees Celsius for about 0.5 minutes to about 30 minutes. In embodiments, the coating solution is spin coated to form a film atop the substrate.

In embodiments, the present disclosure includes a photoresist composition including at least one compound having one or more of the following structures

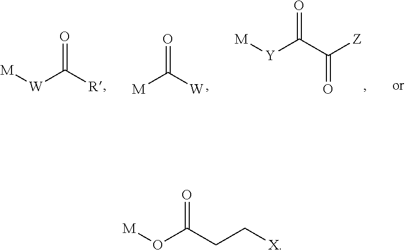

In embodiments, such metal-containing molecular fragments have a formula M=LₙM′, wherein L is a ligand of the present disclosure, and n=0-5, 1-5, or 1, 2, 3, 4, or 5, and M′ is an element selected from the group consisting of Sb, Te, Sn, and Bi. In embodiments, R′ when present, is an element or compound selected from the group of aromatic, aliphatic hydrocarbon, or hydrogen. In embodiments, X when present, is an element or compound selected from the group consisting of F, Cl, Br, I, OCH₂, and CN. In embodiments, W when present, is an element or compound selected from the group consisting of CH₂, NR′″, and S. In embodiments, Y when present, is an element or compound selected from the group consisting of O, CH₂, NR′″, and S. In embodiments, Z when present, is an element or compound selected from the group consisting of R′ and Y—R′.

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where a chiral center exists in a structure, if any, but no specific stereochemistry is shown for that center, both enantiomers, separately or as a mixture, are encompassed by that structure. The methods for the determination of stereochemistry and the separation of stereoisomers are well-known in the art.

In embodiments, any polycyclic compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Non-limiting examples of isotopes suitable for inclusion in the compounds described herein include and are not limited to ²H or deuterium. In one embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In embodiments, the compounds described herein are labeled by other means, including, but not limited to, the use of chromophores or fluorescent moieties, bioluminescent labels, or chemiluminescent labels.

In embodiments, the compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein. Non-limiting examples of forming one or more compounds of the present disclosure are described in the Example section below; however, in embodiments, compounds described herein are synthesized using any suitable procedures starting from compounds that are available from commercial sources or are prepared using procedures described herein.

In embodiments, the present disclosure relates to a method for forming a resist pattern using a lithography composition, including contacting a substrate with a lithography composition of the present disclosure to form a thin radiation sensitive film of the lithography composition atop the substrate.

In embodiments, the present disclosure relates to a method for forming a semiconductor device using one or more lithography compositions of the present disclosure in a photolithography method of the present disclosure. In embodiments, a method for forming a semiconductor device includes contacting a substrate with a lithography composition of the present disclosure to form a thin radiation sensitive film of the lithography composition atop the substrate.

In embodiments, the present disclosure relates to a method for forming an electronic device using the lithography compositions in a photolithography method of the present disclosure. In embodiments, a method for forming an electronic device includes contacting a substrate with a lithography composition of the present disclosure to form a thin radiation sensitive film of the lithography composition atop the substrate.

In embodiments, the present disclosure relates to a substrate and a radiation sensitive coating including a composition of the present disclosure.

In embodiments, the present disclosure related to a substrate including an inorganic semiconductor layer and a radiation sensitive coating material atop a surface, wherein the radiation sensitive coating includes a lithographic composition of the present disclosure. In some embodiments, the radiation coating material or film of the present disclosure can be patterned with EUV light at a wavelength of 13.5 nm in a pattern of 16-nm lines, with a line width roughness of no more than about 4 nm. In embodiments, the radiation sensitive coating material can include metal, such as Te, and can include at least 5 weight percent metal and in other embodiments at least about 20 weight percent metal.

In embodiments, a method for forming an electronic device such as a semiconductor device using the lithography compositions in a photolithography method of the present disclosure. In embodiments, a method for forming an electronic device such as a semiconductor device includes contacting a substrate with a lithography composition of the present disclosure to form a thin radiation sensitive film of the lithography composition atop the substrate. In embodiments, the radiation sensitive film is characterized as amorphous. In embodiments, the radiation sensitive film has a thickness of 10-50 nanometers, such as 20-40 nanometers, such as about 30 nanometers.

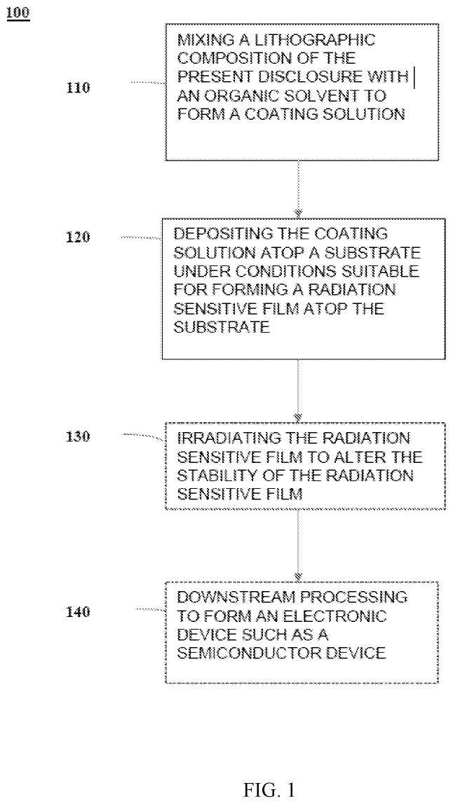

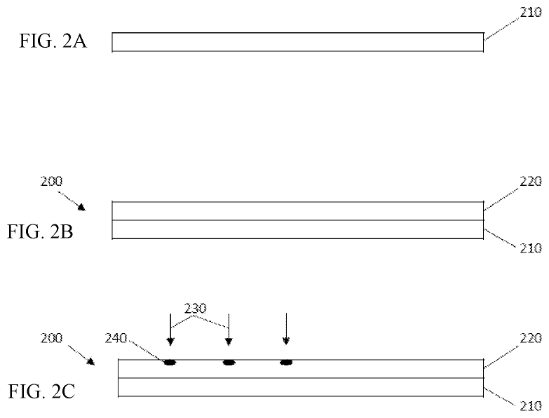

FIG. 1 depicts a flow diagram illustrating a process 100 for forming an electronic device such as a semiconductor device using the lithography compositions in a photolithography method of the present disclosure, which corresponds to FIGS. 2A-2C illustrating schematic cross-sectional views of electronic device 200 such as a semiconductor device at different stages of fabrication. In some embodiments, process 100 is a process flow, and operations 110, 120, and optionally 130 and 140 are individual processes. The process 100 is configured to be performed in an electronic device or semiconductor device manufacturing facility using equipment suitable for depositing layers or coatings atop a substrate.

In embodiments, the process 100 may begin at operation 110 mixing a lithographic composition of the present disclosure with an organic solvent to form a coating solution. For example, a lithographic composition of the present disclosure is formed and provided, and subsequently formed into a solution including a lithographic composition of the present disclosure, including one or more lithographic compounds described above having a general formula [MₐN_{b}O_{c}H_{d}BₑR_{f}X_{g}(WCOR′)ₕ(OR″)ᵢ(WCOCOY)ⱼ(COY)ₖ]^{l} (as described hereinabove), and an organic solvent. In embodiments, the lithographic composition of the present disclosure is provided in an amount sufficient to form a coating solution suitable for forming a film having a predetermined thickness over a substrate.

In embodiments, the process 100 may begin at operation 110 by mixing a lithographic composition of the present disclosure with an organic solvent to form a coating solution. In embodiments, the solvent is an alcohol, an ester, or a mixture thereof. In embodiments, a predetermined lithographic compositions can be dissolved in an organic solvent, e.g., alcohols, aromatic and aliphatic hydrocarbons, esters or combinations thereof. Non-limiting examples of suitable solvents include, for example, aromatic compounds (e.g., xylenes, toluene), ethers (anisole, tetrahydrofuran), esters (propylene glycol monomethyl ether acetate, ethyl acetate, ethyl lactate), alcohols (e.g., 4-methyl-2-propanol, 1-butanol, methanol, isopropyl alcohol, 1-propanol), ketones (e.g., methyl ethyl ketone), mixtures thereof, and the like. In embodiments, organic solvent selection can be influenced by solubility parameters, volatility, flammability, toxicity, viscosity and potential chemical interactions with other processing materials. After the components of the solution are dissolved and combined, the character of the species may change as a result of partial in-situ hydrolysis, hydration, and/or condensation. When the composition of the solution is referenced herein, the reference is to the components as added to the solution, since complex formulations may undergo solvolysis and ligand metathesis, or produce metal polynuclear species in solution that may not be well characterized.

In embodiments, a substrate 210 is provided in the form of silicon, glass, or any suitable material for use as a substrate in electronic device or semiconductor device manufacturing.

Referring to FIG. 1, process 100 includes at process sequence 120 depositing the coating solution atop a substrate under conditions suitable for forming a radiation sensitive film atop the substrate. In embodiments, conditions suitable for forming a radiation sensitive film atop the substrate include conditions suitable for removing the solvent to form a film of lithographic composition in accordance with the present disclosure. In embodiments, removing the solvent may be performed by any method known in the art including heating the mixture under conditions sufficient to evaporate the solvent. For example, the mixture may be heated at or above the boiling point of the solvent disposed within a mixture. For example, where the solvent is ethanol, the mixture can be heated above 78.4 degrees Celsius for a duration and/or under conditions sufficient to evaporate the ethanol from the mixture. In embodiments, after removing the solvent, a coating layer 220 or film including lithographic composition of the present disclosure is formed and disposed atop substrate 210, as shown in FIG. 2B such as wherein the substrate is formed of a preselected materials such as silicon, glass, or the like as described above. In some embodiments, and depending upon needs, the solvent is removed under conditions which permit the formation of an amorphous film of lithographic composition of the present disclosure, wherein the coating layer 220 is characterized as a lithographic film which is photosensitive or reactive to EUV.

Referring back to FIG. 1, process 100 at process sequence 130 optionally includes irradiating the radiation sensitive film such as coating layer 220 to alter the stability of the radiation sensitive film and then develop in a liquid to selectively remove soluble portions prior to downstream processing. In embodiments, the lithographic composition in the form of a film is subject to radiation such as EUV until exposed regions of the film become unstable. In embodiments, a radiation process is applied under conditions suitable to penetrate radiation such as EUV into the coating layer 220 such as a film layer. For example, referring to FIG. 2C, radiation (shown as arrows 230) is applied in an amount and under conditions to alter one or more exposed regions of coating layer 220 atop the substrate 210 to form unstable regions 240.

In embodiments, the coating compositions for forming the resist coatings generally include organometallic compositions of the present disclosure with appropriate radiation sensitive characteristics. For processing into a patternable coating, the lithographic compositions and ligands described herein below, such as ligands 1-4, are generally formed into a solution with a solvent, generally an organic solvent that can be formed into a coating through solution coating or a vapor-based deposition process. The ultimate resist coatings are based on organometallic chemistry, and the lithographic compositions of the present disclosure provide stable solutions with good resist properties. In embodiments, one or more ligands are generally selected to facilitate solution formation and related processing functions. In embodiments, lithographic compositions of the present disclosure with a ligand of the present disclosure can be introduced as a solution to improve the range of compositions that can be formed into stable solutions with the expectation that the coating can provide for patternable coatings with organometallic materials. Compositions of the present disclosure provide desirable patterning properties.

In embodiments, the concentrations of the organometallic materials in the in solutions can be selected to achieve desired physical properties of the solution. In particular, lower concentrations overall can result in desirable properties of the solution for certain coating approaches, such as spin coating, that can achieve thinner coatings using reasonable coating parameters. It can be desirable to use thinner coatings to achieve ultrafine patterning as well as to reduce material costs. In general, the concentration can be selected to be appropriate for the selected coating approach.

In embodiments, coating layer 220 may be formed through deposition and subsequent processing onto a selected substrate. Using the lithographic compositions and coating compositions described herein, some hydrolysis and condensation generally is performed during coating, and may be completed or furthered post coating via subsequent processing steps such as heating in air. In embodiments, a substrate such as substrate 210 generally presents a surface onto which the coating material can be deposited, and the substrate 210 may include a plurality of layers in which the surface relates to an upper most layer. In some embodiments, the substrate surface can be treated to prepare the surface for adhesion of the coating material. Also, the surface can be cleaned and/or smoothed as appropriate. Suitable substrate surfaces may include any reasonable material. Some substrates of particular interest include, for example, silicon wafers, silica substrates, other inorganic materials such as ceramic materials, polymer substrates, such as organic polymers, composites thereof and combinations thereof across a surface and/or in layers of the substrate. Wafers, such as relatively thin cylindrical structures, can be convenient, although any reasonable shaped structure can be used. Polymer substrates or substrates with polymer layers on non-polymer structures can be desirable for certain applications based on their low cost and flexibility, and suitable polymers can be selected based on the relatively low processing temperatures that can be used for the processing of the patternable materials described herein.

In some embodiments, where patterning is performed using radiation, spin coating is a suitable approach to cover the substrate relatively uniformly, although there can be edge effects. In some embodiments, a wafer can be spun at rates from about 400 rpm to about 10,000 rpm. The spinning speed can be adjusted to obtain a desired coating thickness. The spin coating can be performed for times from about 5 seconds to about 5 minutes and in further embodiments from about 15 seconds to about 2 minutes. An initial low speed spin, e.g. at 50 rpm to 250 rpm, can be used to perform an initial bulk spreading of the composition across the substrate. A back side rinse, edge bead removal step or the like can be performed with water or other suitable solvent to remove any edge bead. A person or ordinary skill in the art will recognize that additional ranges of spin coating parameters within the explicit ranges above are contemplated and are within the present disclosure.

The thickness of the coating generally can be a function of the coating solution concentration, viscosity and the spin speed for spin coating. For other coating processes, the thickness can generally also be adjusted through the selection of the coating parameters. In some embodiments, it can be desirable to use a thin coating to facilitate formation of small and highly resolved features in the subsequent patterning process. For example, the coating materials after drying can have an average thickness of no more than about 200 nanometers (nm), in other embodiments no more than about 150 nm, in further embodiments no more than about 80 nanometers (nm), in additional embodiments from about 5 nanometers (nm) to about 50 nm, in other embodiments from about 5 nm to about 40 nm and in some embodiments from about 8 nm to about 40 nm.

In embodiments, the coating process itself can result in the evaporation of a portion of the solvent since many coating processes form droplets or other forms of the coating material with larger surface areas and/or movement of the solution that stimulates evaporation. The loss of solvent tends to increase the viscosity of the coating material as the concentration of the species in the material increases. An objective during the coating process can be to remove sufficient solvent to stabilize the coating material for further processing. Reactive species may condense during coating or subsequent heating to forming a coating material.

Following drying, the coating material can be finely patterned using radiation. In embodiments, the absorption of the radiation results in energy that can break the bonds between the metal and alkyl ligands so that at least some of the alkyl ligands are no longer available to stabilize the material. Radiolysis products, including alkyl ligands or fragments may diffuse out of the film, or not, depending on process variables and the identity of such products. In embodiments, with the absorption of a sufficient amount of radiation, the exposed coating material condenses. The radiation generally can be delivered according to a selected pattern. In embodiments, the radiation pattern is transferred to a corresponding pattern or latent image in the coating material with irradiated areas and un-irradiated areas. The irradiated areas include chemically altered coating material, and the un-irradiated areas include generally the as-formed coating material. As noted below, very smooth edges can be formed upon development of the coating material with the removal of the un-irradiated coating material or alternatively with selective removal of the irradiated coating material.

Radiation generally can be directed to the coated substrate through a mask or a radiation beam can be controllably scanned across the substrate. In general, the radiation can include electromagnetic radiation, an electron-beam (beta radiation), or other suitable radiation. In embodiments, electromagnetic radiation can have a desired wavelength or range of wavelengths, such as visible radiation, ultraviolet radiation or x-ray radiation. The resolution achievable for the radiation pattern is generally dependent on the radiation wavelength, and a higher resolution pattern generally can be achieved with shorter wavelength radiation. Thus, it can be desirable to use ultraviolet light, x-ray radiation or an electron-beam to achieve particularly high-resolution patterns.

In embodiments, ultraviolet light is provided which extends between wavelengths of greater than or equal 100 nm and less than 400 nm. In embodiments, a krypton fluoride laser can be used as a source for 248 nm ultraviolet light. The ultraviolet range can be subdivided in several ways, such as extreme ultraviolet (EUV) from greater than or equal 10 nm to less than 121 nm and far ultraviolet (FUV) from greater than or equal to 122 nm to less than 200 nm. In embodiments, EUV light has been used for lithography at 13.5 nm, and this light is generated from a Xe or Sn plasma source excited using high energy lasers or discharge pulses.

The amount of electromagnetic radiation can be characterized by a fluence or dose which is defined by the integrated radiative flux over the exposure time. A person of ordinary skill in the art will recognize the ranges of radiation fluences within the explicit ranges above are contemplated and are within the present disclosure.

Based on the design of the coating material, there can be a large contrast of material properties between the irradiated regions that have condensed coating material and the un-irradiated, coating material with substantially intact organic ligands.

In embodiments, following exposure with radiation, the coating material is patterned with irradiated regions and un-irradiated regions. Referring to FIG. 2C, a patterned structure is shown including a substrate 210, a thin film 220 and patterned coating material 240 or regions of instability. In embodiments, patterned coating material includes region 240 of irradiated coating material and uncondensed regions un-irradiated coating material. The pattern formed by condensed regions and uncondensed regions represent a latent image into the coating material, and the development of the latent image is performed as is known in the art.

In embodiments, development of the image involves the contact of the patterned coating material including the latent image to a developer composition to remove either the un-irradiated coating material to form the negative image or the irradiated coating to form the positive image. Using the resist materials described herein, effective negative patterning or positive patterning generally can be performed with desirable resolution using appropriate developing solutions, and generally based on the same coating.

Based on the improved process described in the Examples below, the improved properties of the coating material can be correspondingly characterized. For example, a substrate including an inorganic semiconductor layer and a radiation sensitive coating material of the present disclosure along a surface can be subjected to patterning with EUV light at a wavelength of 13.5 nm in a pattern of 16-nm lines on a 32-nm pitch. In embodiments, a surface can be subjected to patterning with EUV light at a wavelength of 13.5 nm in a pattern of 32-nm lines on a 64-nm pitch. To evaluate the coating material the dose to achieve a critical dimension of 16 nm can be evaluated along with the achievable line width roughness (LWR). In embodiments, the coatings can achieve a critical dimension of 16 nm with a dose from about 20 mJ/cm2 to about 120 mJ/cm2 with a line width roughness of no more than about 4 nm. Resist critical dimension (CD) and line-width-roughness (LWR) were extracted from SEM images.

In further embodiments, the improved patterning capability can be expressed in terms of the dose-to-gel value. A structure comprising a substrate and a radiation sensitive coating comprising an alkyl metal oxide hydroxide can have a dose-to-gel (D_{g}) of no more than about 60 mJ/cm² and in further embodiments from about 10 mJ/cm² to about 40 mJ/cm². Evaluation of dose-to-gel is explained in the Examples below.

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present disclosure.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

In embodiments, the present disclosure includes one or more amorphous photoresist film compositions including one or more ligands, such as a first ligand, second ligand, third ligand, and/or fourth ligand of the present disclosure. In embodiments, a first ligand is one or more carboxylate ligands (e.g. —OC═OR′) expanded by inserting other molecular fragments such as CH₂, NR′″, or S between the metal and the carbonyl. In embodiments, suitable molecular fragments are less electronegative than the oxygen they replace yet retain the ability to stabilize unstable intermediates such as radicals, anions of cations. In embodiments, a first ligand of the present disclosure is characterized by the formula

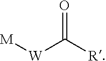

In embodiments, W is each individually one of CH₂, NR′″, S, R′, —XR′, or

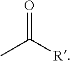

In embodiments, R′ is aromatic, aliphatic hydrocarbon, or H. In embodiments, R′″ is H or R′. In embodiments, a metal containing molecular fragment of the present disclosure is characterized as M=LnM′, wherein n=0-5, or 1-5, L=one or more ligands (such as W—COR′) of the present disclosure, and M′ is Sb, Te, Sn, or Bi. For clarity M is not the ligand, however it depicts where the ligand attached to the metal. In embodiments, X=F, Cl, Br, I, OCH₃, or CN. Non-limiting examples of a first ligand of the present disclosure include:

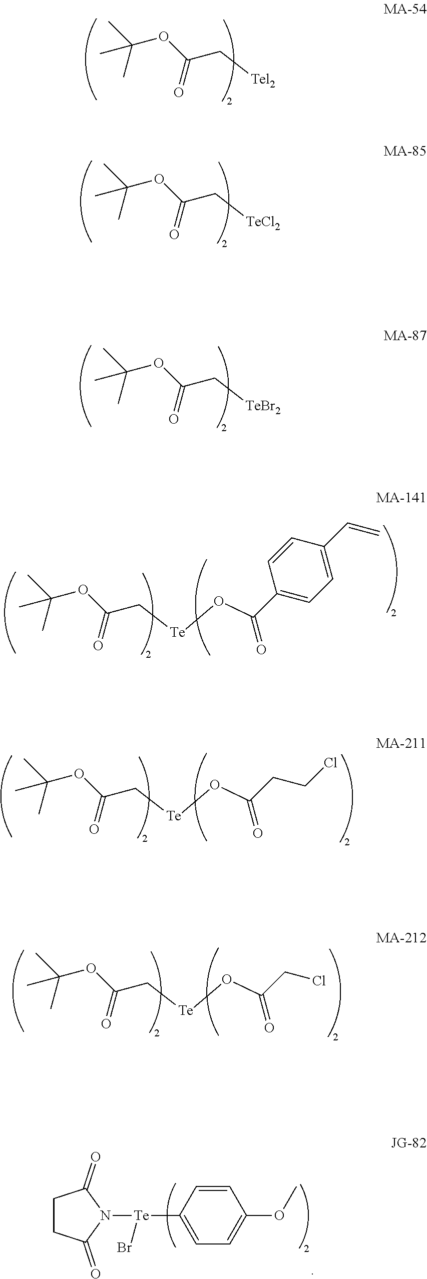

In some embodiments, a first ligand of the present disclosure is characterized as WCOR′ as described hereinabove.

In embodiments, a second ligand of the present disclosure includes one or more ligands with good reactivity such as an oxalate ligand. In embodiments, a second ligand of the present disclosure shares structural feature with oxalate (e.g., adjacent carbonyl groups) and is readily incorporated into molecular structures to form stable complexes. In embodiments, the a second ligand of the present disclosure is characterized by the formula:

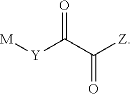

In embodiments, Z=R′, Y—R′. In embodiments, Y=O, CH₂, NR′″, or S. In some embodiments, metal-containing molecular fragments suitable for use in forming compositions of the present disclosure have a formula, M=LnM′, wherein n=0-5 such as 1-5, L=one or more ligands of the present disclosure, and M′ is Sb, Te, Sn, or Bi. Non-limiting examples of a second ligand of the present disclosure include:

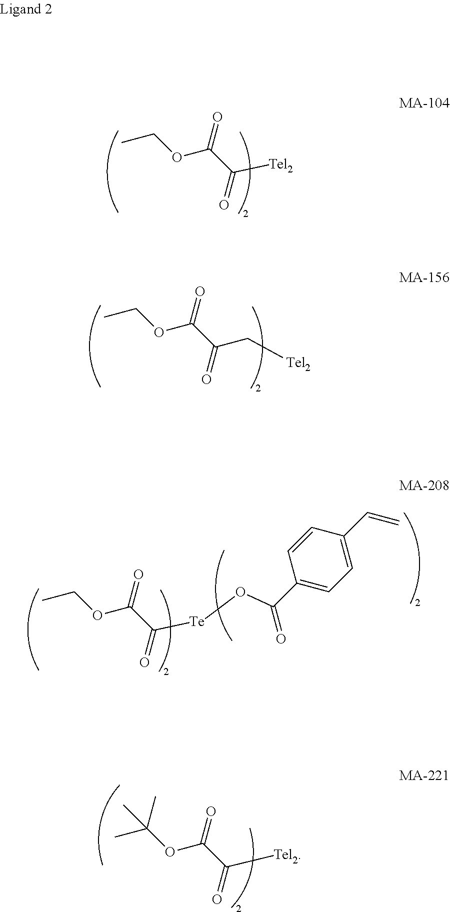

In some embodiments, a second ligand of the present disclosure is characterized as WCOCOY as described hereinabove.

In embodiments, a third ligand of the present disclosure is characterized by the formula:

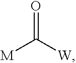

wherein W is each individually one of CH2, NR′″, or S; and M=LnM′, wherein n=0-5, L=one or more ligands of the present disclosure, and M′ is Sb, Te, Sn, or Bi. Non-limiting examples of a third ligand of the present disclosure include:

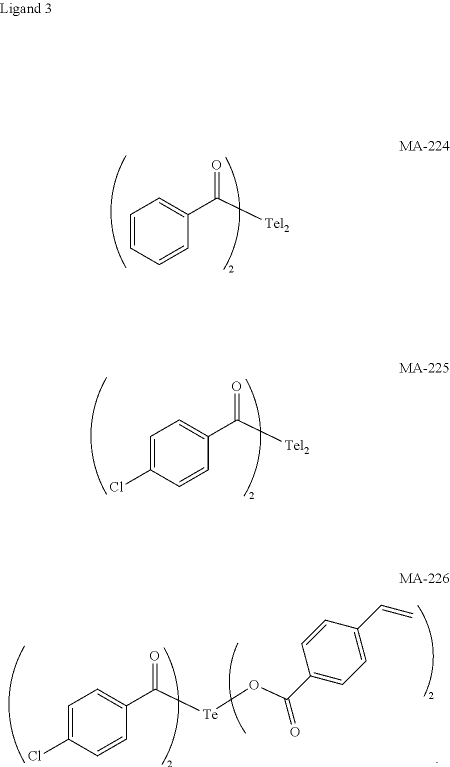

In embodiments, a third ligand of the present disclosure is characterized as COY as described herein above.

In embodiments, a fourth ligand of the present disclosure is characterized by the formula:

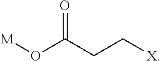

In some embodiments, metal-containing molecular fragments suitable for use in forming compositions of the present disclosure have a formula, M=LnM′, wherein n=0-5 or 1-5, L=one or more ligands of the present disclosure, and M′ is Sb, Te, Sn, or Bi, and X=F, Cl, Br, I, OCH₃, or CN. Non-limiting examples of a fourth ligand of the present disclosure include:

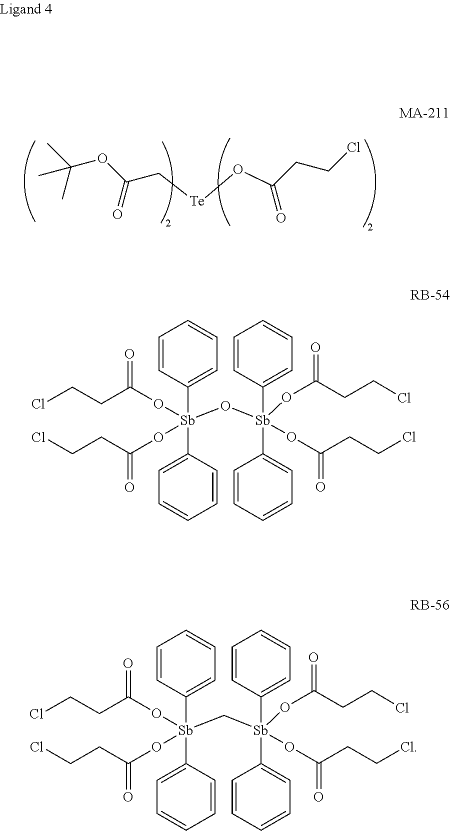

In some embodiments, the fourth ligand of the present disclosure is characterized as WCOR′ as described herein above.

Type 1: Iodate Cluster. Preparation of Iodate cluster: The reaction was carried out under a nitrogen atmosphere with the use of standard Schlenk techniques. Periodic acid (1.0 equiv.) was added to a solution of methanol (30 mL) and potassium hydroxide (4.0 equiv.). The mixture was stirred for 0.5 h. After the addition of metal salt (R₂NX₂ where N=Te or Sn; 2.0 equiv.), the mixture was stirred at 50° C. for 12 h and then filtered. The solvent was gradually removed by evaporation under vacuum until a solid product was obtained. The solid was then recrystallized from diethyl ether, and colorless crystals of complex were recovered.

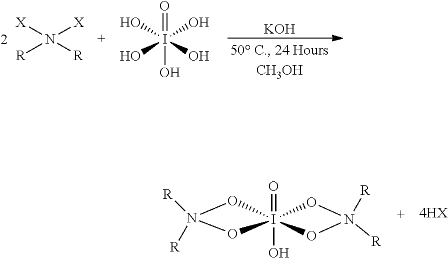

### Where

X=I, Cl
N=Te, Sn
R=CH₃OC₆H₄, CH₃OC₆H₄CO, tBuOCOCO, C₆H₅CO, C₆H₅CH₂

### Specific Example

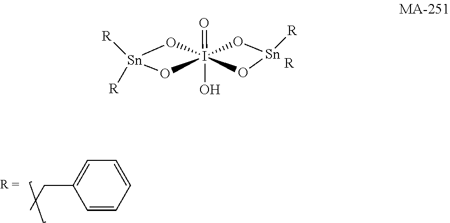

Iodate Cluster Synthesis: R=C₆H₅OCH₃, CH₃OC₆H₄CO, C(CH₃)₃OCOCO, C₅H₄CO are MA-192, MA-218, MA-227, MA-229, and R=C₆H₅CH₂, M=Sn is MA-251 respectively.

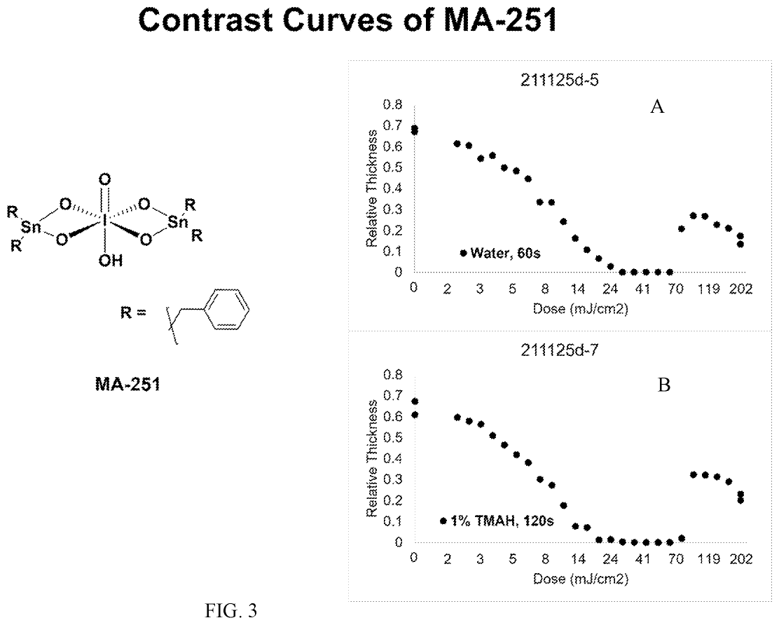

Contrast Curves for Periodate Cluster. A dilute solution (2 wt %) of MA-251 in 1,4-dioxane was spin coated at 2000 rpm onto a 100 mm silicon wafer followed by a bake at 60° C. for 60 s, yielded an amorphous film. Exposure of the film to a range of 26 doses from 2 to 200 mJ/cm² of extreme ultraviolet (EUV, 13.5 nm) light under vacuum, followed by development in (A) water or a (B) 2.6 mM solution of tetramethyl ammonium (TMAH) in water for 60 or 120 s, respectively, yielded an array of dots of variable thicknesses. Measurement of the thickness of the exposed and unexposed areas of the wafer using ellipsometry yielded the two contrast curves shown in FIG. 3.

### Type 2: Oxygen Bridge Metal Complexes.

Step 1: Metal halide salt (R₃SbX₂; 2.0 equiv.) was added to a round bottom flask (25 mL) and dissolved in acetone (15 mL). Sodium hydroxide (1.0 equiv, 71 mg) was added in another round bottom flask (25 mL) and dissolved in methanol (15 mL). Both solutions were added were added to a third round bottom flask (50 mL) equipped with a magnetic stir bar. The flask was then connected to a reflux condenser and placed in an oil bath. The mixture was stirred for 2 hours at 70° C. Then, the mixture was filtered, and the solvent was removed via rotary evaporator. The residue was then extracted with dichloromethane (2×30 mL). The combined organic fractions were extracted with water to remove sodium halide salts, dried over Na₂SO₄ and reconcentrated under vacuum to give the desired product (O(SbR₃X)₂) as a white solid.

Step 2: Bis(metal halide)oxide (1.0 equiv), potassium carboxylate (e.g. ClCH₂CH₂CO₂K; 2.0 equiv.), dichloromethane (20 mL) and water (10 mL) were added to round bottom flask (50 mL) equipped with a magnetic stir bar. The flask was then connected to a reflux condenser and placed in an oil bath. The mixture was stirred for 3 hours at 50° C. Then, the mixture was filtered, and the solvent was removed using a rotary evaporator. The residue was then extracted with dichloromethane (2×30 mL). The combined organic fractions were extracted with water to remove sodium halide salts, dried over Na₂SO₄ and reconcentrated under vacuum to give the desired product (Oxygen Bridge Metal Complex) as a white solid. RB-54: Yield 45%, m.p. 208° C., ¹H NMR (500 MHz, CDCl₃): δ 2.57 (t, 4H), 3.56 (t, 4H), 7.5-8.3 (7.63 (dd, 18H), (d, 8.11 (dddd, 12H)).

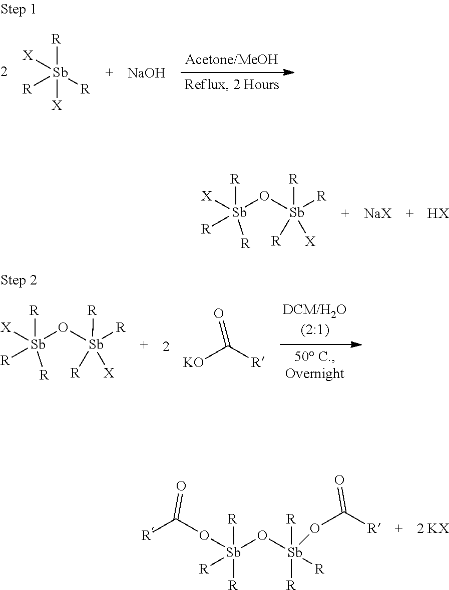

Where X=Cl, I Where R′=CH₂CH₂Cl, CH₂Cl, C₆H₄CH═CH₂ R=C₆H₅, CH(CH₃)₂, C₆H₄CH═CH₂

### Example

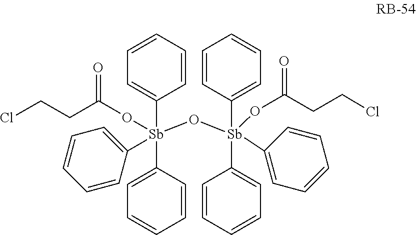

Oxygen Metal Complex Synthesis: where R=C₆H₅ and R′=CH₂CH₂Cl, CH₂Cl are RB-54, and RB-104, respectively. R=C₆H₄CHCH₂ and R′=CH₂CH₂Cl is RB-124, When R=CH(CH₃)₂ and R′=—CH₂CH₂Cl, C₆H₄CHCH₂ are RB-109 and RB-110, respectively.

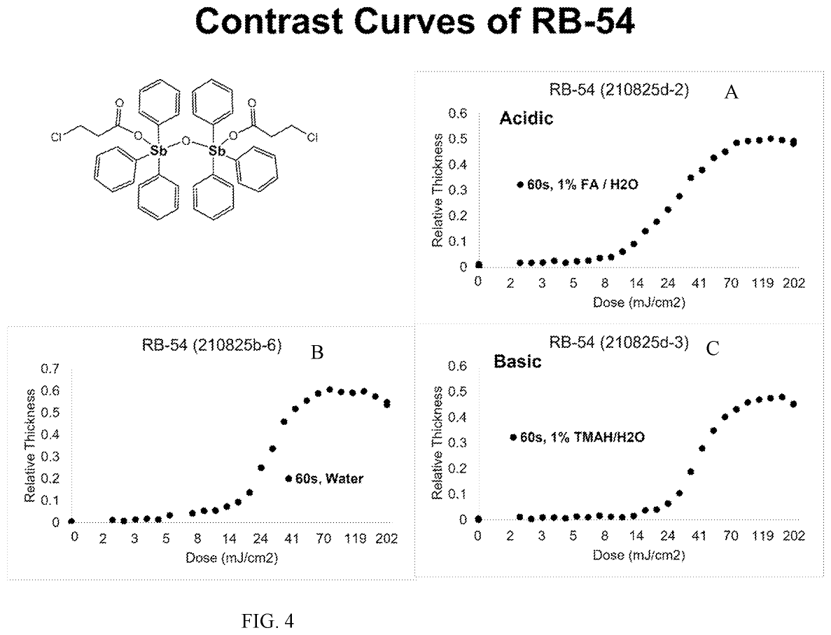

Contrast Curves of RB-54. A dilute solution (1.5 wt %) of RB-54 in 1,4-dioxane was spin coated at 2000 rpm onto a 100 mm silicon wafer followed by a bake at 60° C. for 60 s, yielded an amorphous 120-nm thick film. Exposure of the film to a range of 26 doses from 2 to 200 mJ/cm² of extreme ultraviolet (EUV, 13.5 nm) light under vacuum, followed by development in (A) 1 wt % formic acid in water, (B) water, or (C) 2.6 mM solution of tetramethyl ammonium (TMAH) in water for 60 s, yielded arrays of dots of variable thicknesses for each sample. Measurement of the thickness of the exposed and unexposed areas of the wafer using ellipsometry yielded the three contrast curves shown in FIG. 4.

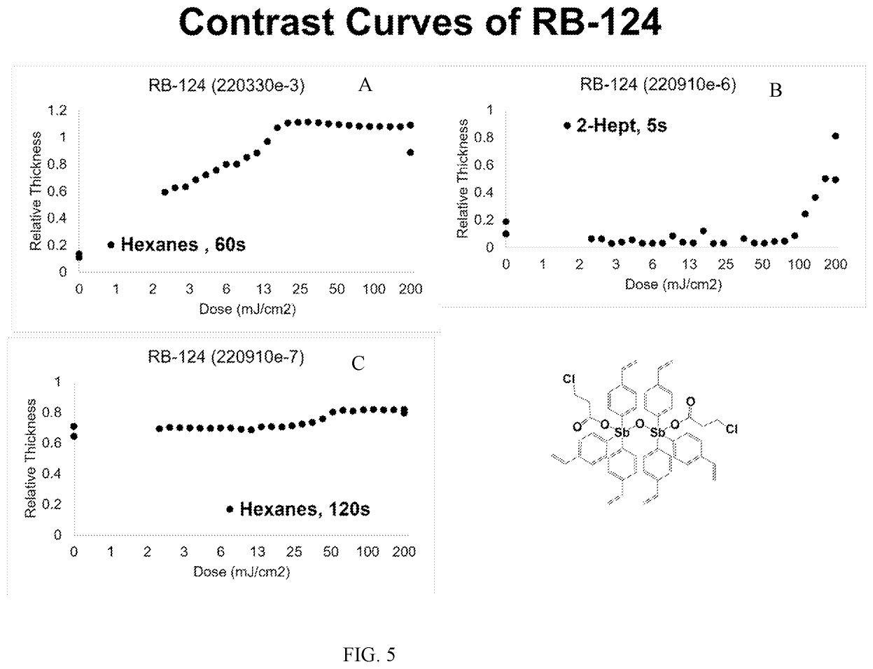

Contrast Curves of RB-124. A dilute solution (2 wt %) of RB-124 in dichloroethane was spin coated at 2000 rpm onto a 100 mm silicon wafer followed by a bake at 60° C. for 60 s, yielded an amorphous 46-nm thick film. Exposure of the film to a range of 26 doses from 2 to 200 mJ/cm² of extreme ultraviolet (EUV, 13.5 nm) light under vacuum, followed by development in (A) hexanes or (B) 2-hepatanone for 60 s or 5 s, respectively, yielded arrays of dots of variable thicknesses for each sample. Measurement of the thickness of the exposed and unexposed areas of the wafer using ellipsometry yielded the three contrast curves shown FIG. 5.

### Type 3: Bridged Metal Carboxylate Complex (Prophetic)

Step 1: Metal halide salt (R_{f/2}MX₂; 2.0 equiv.) is added to a round bottom flask (25 mL) and is dissolved in acetone (15 mL). Bridge precursor (e.g. KO₂CCH═CHCO₂K; 1.0 equiv.) is added in another round bottom flask (25 mL) and dissolved in methanol (15 mL). In a new round bottom flask (50 mL), both solutions are added followed by a magnetic stir bar. The flask is then connected to a reflux condenser and placed in an oil bath. The mixture is stirred for 2 hours at 70° C. Then, the mixture is filtered, and the solvent is removed via rotary evaporator. The residue is extracted with dichloromethane (2×30 mL). The combined organic fractions are extracted with water to remove sodium halide salts, dried over Na₂SO₄ and reconcentrated under vacuum to give the desired product (O(MR_{f/2}X)₂) as a white solid.

Step 2: The bis(metal halide)bridge complex (e.g. O(MR_{f/2}X)₂; 1.0 equiv.), the potassium carboxylate (e.g. CH₂=CHC₆H₄CO₂K; 2.0 equiv.), dichloromethane (20 mL) and water (10 mL) were added to round bottom flask (50 mL) equipped with a magnetic stir bar. The flask is then connected to a reflux condenser and placed in an oil bath. The mixture is stirred for 3 hours at 50° C. Then, the mixture is filtered, and the solvent is removed via rotary evaporator. The residue is extracted with dichloromethane (2×30 mL). The combined organic fractions are extracted with water to remove sodium halide salts, dried over Na₂SO₄ and reconcentrated under vacuum to give the desired product (Bridged Metal Carboxylate Complex) as a white solid.

### Synthesis of the Bridged Metal Carboxylate Complex:

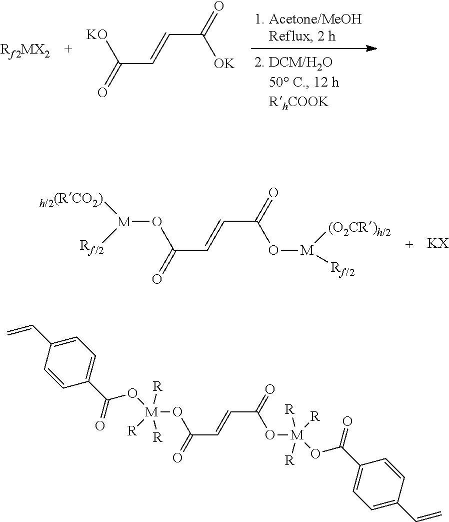

### Type 4: Tellurium Acid Cluster.

Preparation of Tellurium Acid Cluster: The reaction was carried out under a nitrogen atmosphere with the use of standard Schlenk techniques. The Telluric acid (1.0 equiv.) was added to the solution of methanol (30 mL) together with potassium hydroxide (4.0 equiv.), and the mixture was stirred for 0.5 h. After the addition of metal salt (R₂NX₂ where N=Sb or Sn; 2.0 equiv.),² the mixture was stirred at 50° C. for 12 h and then filtered. The solvent was gradually removed by evaporation under vacuum until a solid product was obtained. The solid was then recrystallized from diethyl ether, and colorless crystals of complex were recovered.

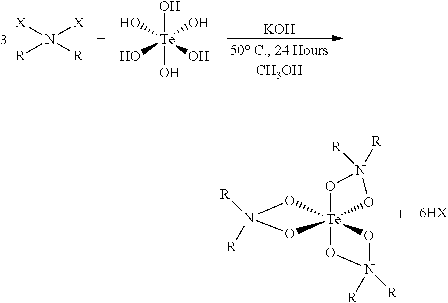

Where
   X=I, Br, Cl
   N=Sb, Sn
   R=C₆H₅CH₂, C₆H₁₁, C₆H₄CH═CH₂, C(CH₃)₂=CH₂, CH₄C₂H₅

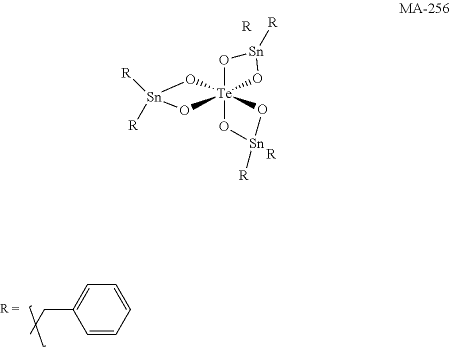

Telluric Acid Cluster Synthesis: R=C₆H₁₁, C₆H₄CH═CH₂, C(CH₃)₂=CH₂, C₆H₄C₂H₅ and M=Sb are NU-158, NU-87, NU-82, NU-160 and R=C₆H₅CH₂, M=Sn is MA-256 respectively.

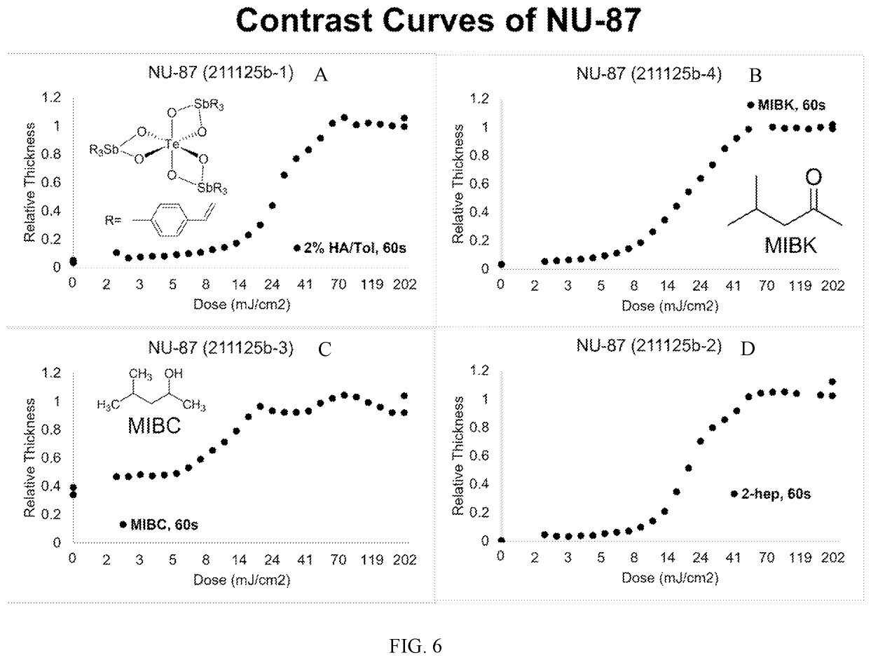

Contrast Curves of NU-87. A dilute solution (1.5 wt %) of NU-87 in dichloroethane was spin coated at 2000 rpm onto a 100 mm silicon wafer followed by a bake at 60° C. for 60 s, yielded an amorphous film. Exposure of the film to a range of 26 doses from 2 to 200 mJ/cm² of extreme ultraviolet (EUV, 13.5 nm) light under vacuum, followed by development in (A) 2 wt % hexanoic acid in toluene, (B) methyl isobutyl ketone (MIBK), (C) 4-methyl-2-pentanol (MIBC) or (D) 2-heptanone all for 60 s, yielded arrays of dots of variable thicknesses for each sample. Measurement of the thickness of the exposed and unexposed areas of the wafer using ellipsometry yielded the three contrast curves shown in FIG. 6.

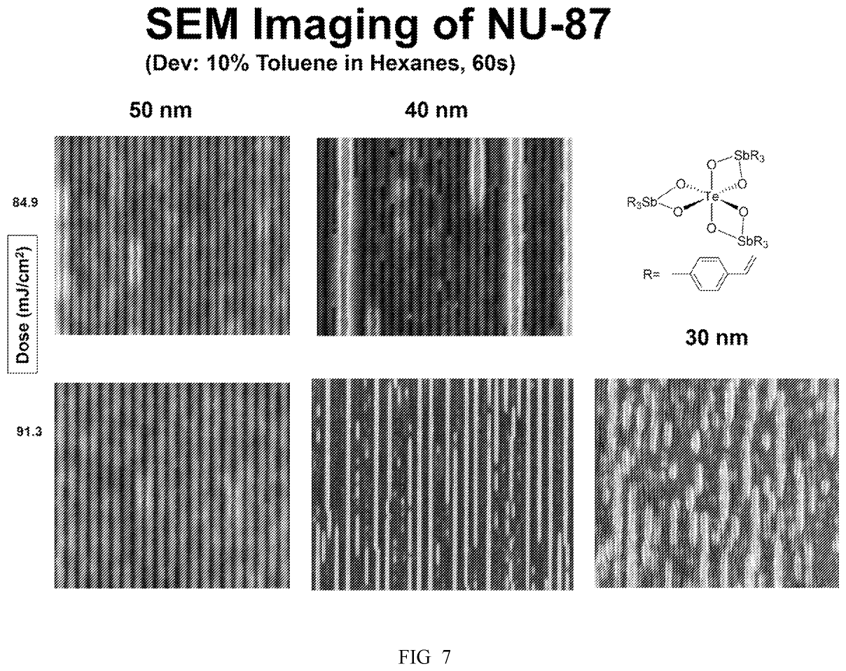

Imaging of NU-87. An amorphous film of NU-87 was exposed using extreme ultraviolet (EUV, 13.5 light via interference lithography at the XIL-II beam line Paul Shirrer Institute. Development in 10% toluene in hexanes for 60 s produced 50 and 40-nm lines as shown in the SEM images of FIG. 7.

### Type 5: Tellurium Acid Clusters.

Preparation of Tellurium Acid Clusters. The reaction was carried out under a nitrogen atmosphere with the use of standard Schlenk techniques. The Telluric acid (1.0 equiv.) was added to the solution of methanol (30 mL) together with potassium hydroxide (4.0 equiv.) and the mixture was stirred for 0.5 h. After the addition of metal salt (R₂TeX₂; 2.0 equiv.), the mixture was stirred at 50° C. for 12 h and then filtered. The solvent was gradually removed by evaporation under vacuum until a solid product was obtained. The solid was then recrystallized from diethyl ether, and colorless crystals of complex were recovered.

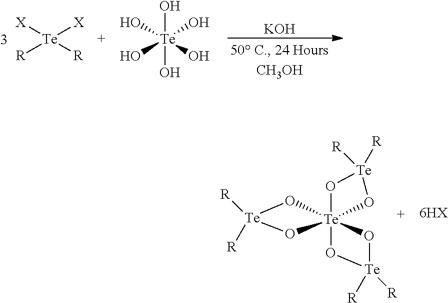

Where
   X=I, Cl
   M=Te
   R=C₆H₅OCH₃, COC₆H₅, CH₂C₆H₅, C(CH₃)₃O₂CCH₂ and C₆H₄C₂H₃

### Example

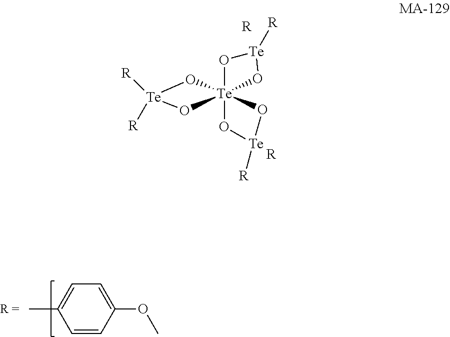

Telluric Acid Cluster: R=C₆H₅OCH₃, COC₆H₅, CH₂C₆H₅ are MA-129, MA-176, MA-216, MA-129 and R=C(CH₃)₃O₂CCH₂ and C₆H₄C₂H₃ is MA-207 respectively.

### Type 6: Carbon Bridge Metal Complexes.

Step 1: Metal precursor (R₃M, 1.0 equiv.) was added to a two neck round bottom flask (250 mL) equipped with a magnetic stir bar. The flask was then sealed with a septa and a glass stopper. The entire setup was then purged with nitrogen for 2 hours to keep the reaction conditions as dry (free of moisture) as possible. Dry tetrahydrofuran (100 mL) was then added through the septa via syringe and stirring was applied for 5 mins to dissolve the solid. High pressure nitrogen was applied through the septa and the glass stopper was removed to add pellets of sodium metal (2.2 equiv., 1.07 g) to the flask. The flask was then resealed with the glass stopper and the high-pressure nitrogen was removed from the flask. The mixture was stirred for 24 hours at room temperature. At the endpoint of the reaction the solution became dark red. While stirring, ammonium chloride (1.0 equiv., 1.12 g) was added as a suspension in dry tetrahydrofuran (20 mL) via syringe to consume the phenyl sodium that was produced. Dichloromethane (0.5 equiv., 1.5 mL) was added dropwise until the color of the solution disappeared. Then, the excess sodium was carefully removed and placed on isopropyl alcohol to quench. The solvent was removed by vacuum via the rotary evaporator. Water (100 mL), ether (100 mL) and benzene (50 mL) were added to dissolve the residue and was placed in a separatory funnel. The combined organic layers were collected, separated, and dried with anhydrous sodium sulfate. The solvent was distilled off under reduced pressure, and the residual solid was recrystallized from acetone to give colorless or slightly yellow crystals of R₂MCH₂MR₂. This compound is stable to air in the solid state but oxidizes slowly in solution.

Step 2: R₂MCH₂MR₂ (1.0 equiv.) was added to a round bottom flask (100 mL) with a magnetic stir bar followed by tetrahydrofuran (15 mL). The flask was then sealed with a septa. While stirring at room temperature, a solution of iodine (2.0 equiv., 1.345 g) in tetrahydrofuran (40 mL) was added dropwise via syringe until the solution turned pink. Then, the solvent was distilled off under reduced pressure with the rotary evaporator which made a slightly yellow powder (CH₂(M R₂I₂)₂).

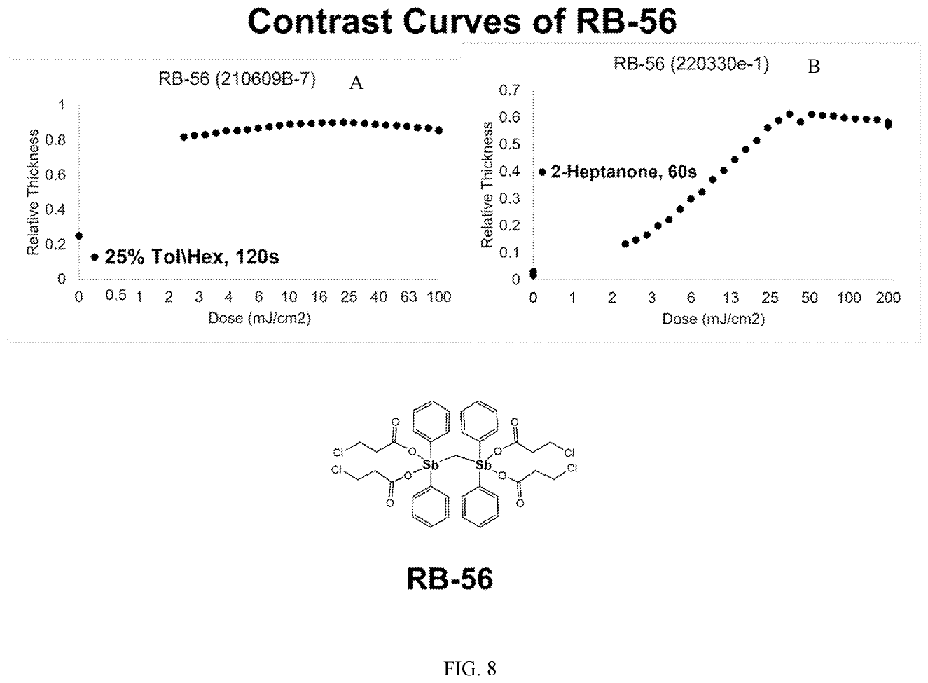

Step 3: Carbon-bridge metal salt [(CH2(M R₂I₂)₂), 1.0 equiv.] and potassium carboxylate (4.0 equiv.) was added to round bottom flask (50 mL) with a stir bar followed by dichloromethane (20 mL) and water (10 mL). The flask was then connected to a reflux condenser and placed in an oil bath. The mixture was stirred for 3 hours at 50° C. Then, the mixture was filtered, and the solvent was removed via rotary evaporator. The residue was then extracted with dichloromethane (2×30 mL). The combined organic fractions were extracted with water to remove sodium halide salts, dried over Na₂SO₄ and reconcentrated under vacuum to give the desired product (Carbon Bridge Metal Complex) as a white solid. RB-56: H NMR (500 MHz, (CD3)2SO): δ 1.36 (s, 2H), 2.57 (t, 4H), 3.56 (t, 4H), 7.77 (dd, 12H), 8.01 (dd, 8H)). See contrast curve of RB-56 in FIG. 8.

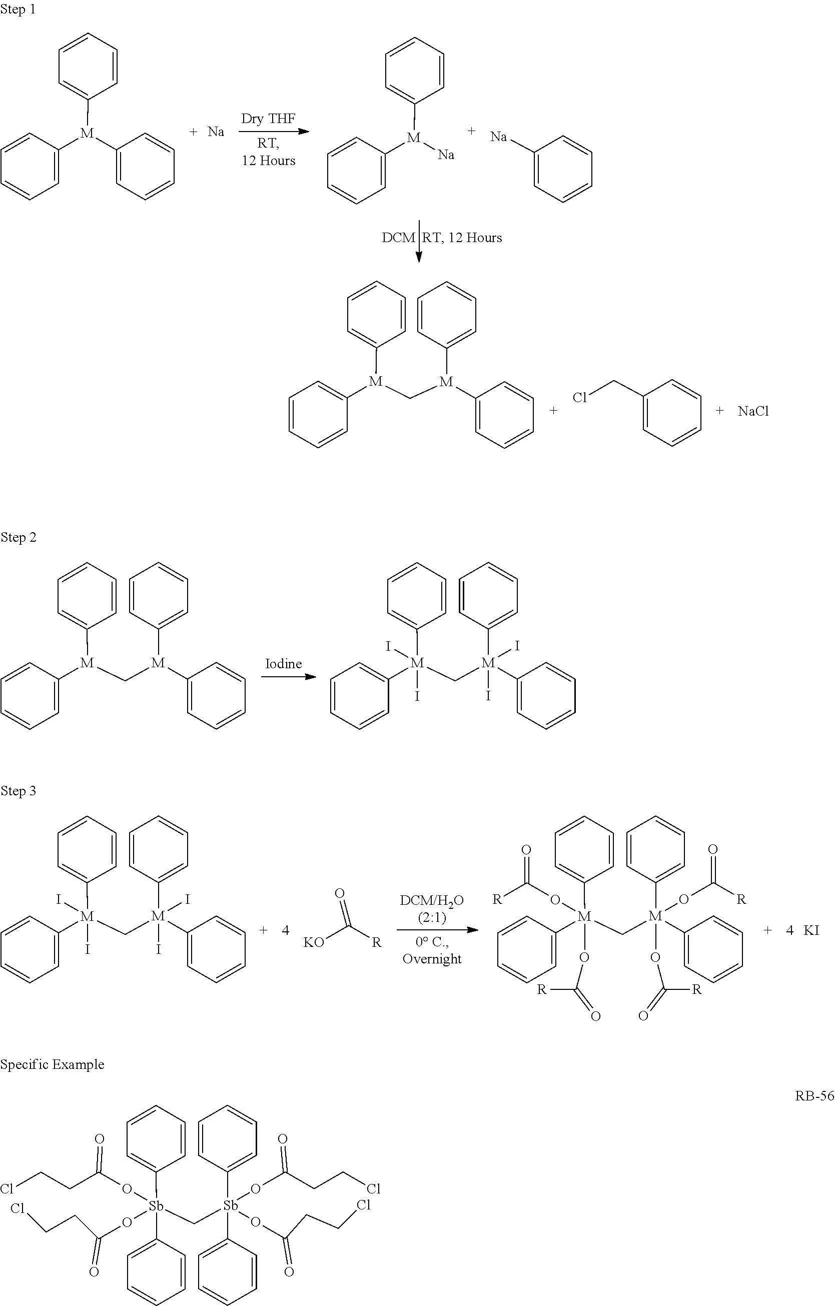

Oxygen Metal Complex Synthesis where R=CH₂CH₂Cl, M=Sb is RB-56 and R=C₆H₄CHCH₂, M=Sn is RB-129 respectively.

Contrast Curves of RB-56. A dilute solution (1.5 wt %) of RB-56 in 1,4-dioxane was spin coated at 2000 rpm onto a 100 mm silicon wafer followed by a bake at 60° C. for 60 s, yielded a 45-nm thick amorphous film. Exposure of the film to a range of 26 doses from 2 to 200 mJ/cm² of extreme ultraviolet (EUV, 13.5 nm) light under vacuum, followed by development in (A) 25% toluene in hexanes or (B) 2-heptanone for 120 s and 60 s, respectively, yielded arrays of dots of variable thicknesses for each sample. Measurement of the thickness of the exposed and unexposed areas of the wafer using ellipsometry yielded the three contrast curves shown in FIG. 8.

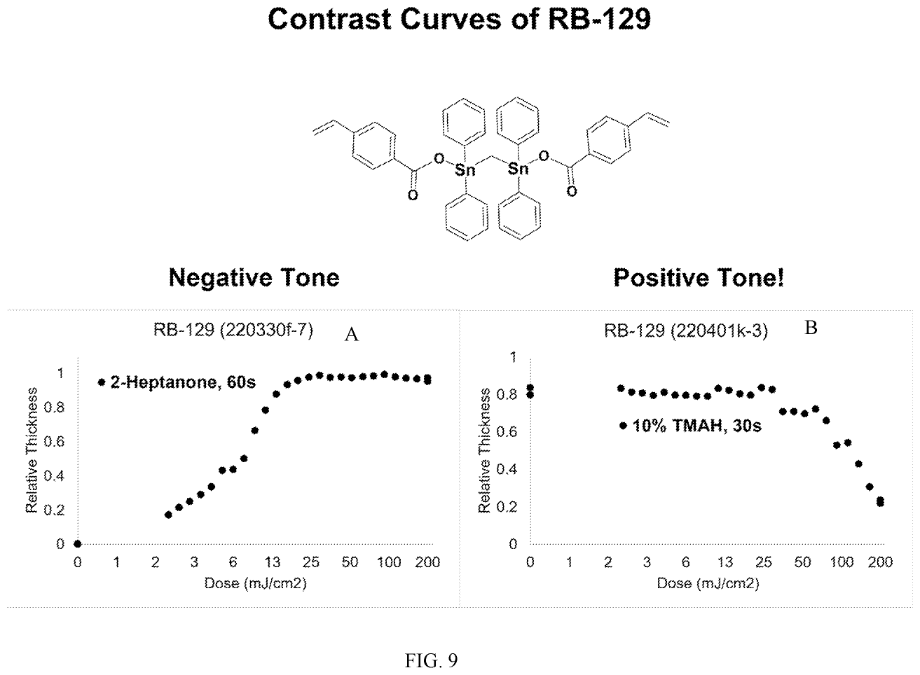

Contrast Curves of RB-129. A dilute solution (1.5 wt %) of RB-129 in 1,4-dioxane was spin coated at 2000 rpm onto a 100 mm silicon wafer followed by a bake at 60° C. for 60 s, yielding a 66-nm thick amorphous film. Exposure of the film to a range of 26 doses from 2 to 200 mJ/cm² of extreme ultraviolet (EUV, 13.5 nm) light under vacuum, followed by development in (A) 2-heptanone or (B) 26 mM solution of tetramethyl ammonium (TMAH) in water for 60 and 30 s, respectively, yielded arrays of dots of variable thicknesses for each sample. Measurement of the thickness of the exposed and unexposed areas of the wafer using ellipsometry yielded the three contrast curves shown in FIG. 9.

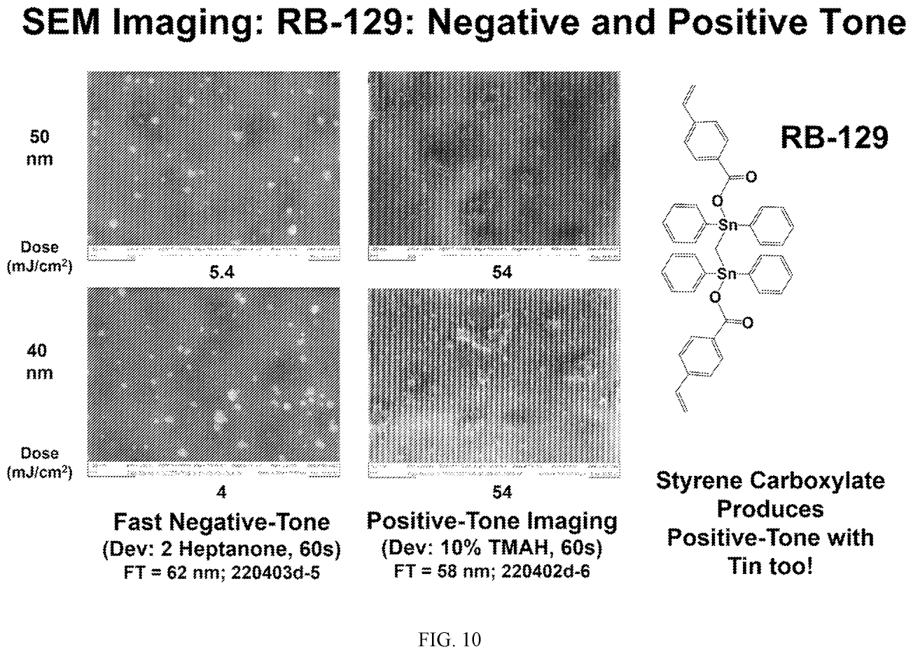

Imaging of RB-129. An amorphous film of RB-129 was exposed using extreme ultraviolet (EUV), 13.5 light via interference lithography at the XIL-II beam line Paul Shirrer Institute. Development in 2-heptanone for 60 s yielded 50 and 40-nm negative-tone lines, and development in 26 mM solution of tetramethyl ammonium (TMAH) in water for 60 s yielded 50 and 40-nm positive-tone lines as shown in the SEM images of FIG. 10.

### Type 7: Bridge Metal Carboxylate Complex. (Prophetic)

Step 1: The compound triphenyltinchloride (1.0 equiv.) is added to a two neck round bottom flask (250 mL) equipped with a magnetic stir bar. The flask is then sealed with a septa and a glass stopper. The entire setup is then purged with nitrogen for 2 hours to keep the reaction conditions as dry (free of moisture) as possible. Dry tetrahydrofuran (100 mL) is then added through the septa via syringe and stirring is applied for 5 mins to dissolve the solid. High pressure nitrogen is applied through the septa and the glass stopper is removed to add pellets of sodium metal (2.5 equiv.) to the flask. The flask is then resealed with the glass stopper and the high-pressure nitrogen is removed from the flask. The mixture is stirred for 24 hours at room temperature. While stirring, 1,3-Dibromopropane (0.5 equiv.)) is added dropwise. The mixture is stirred for an additional 15 minutes. Then, the solvent is removed via rotary evaporator. The resulting solid is extracted with sequential additions of water (100 mL), ether (100 mL) and benzene (50 mL). The combined extracts are collected, separated, and dried with anhydrous sodium sulfate. The solvent is removed under reduced pressure yielding a white powder (bis(triphenyltin)propane).

Step 2: The compound, bis(triphenyltin)propane (1.0 equiv.) and benzene (70 mL) is added to a round bottom flask (100 mL) equipped with a magnetic stir bar. The reaction is then placed under nitrogen and stirred at room temperature. A solution of iodine (2.0 equiv.) in methanol (20 mL) is added via syringe, the mixture is stirred for 48 hours. Then, the solvent is distilled off under reduced pressure via rotary evaporator yielding a slightly pink powder (bis(diphenyltin iodide)propane).

Step 3: The compound, bis(diphenyltin iodide)propane (1.0 equiv.) and the potassium carboxylate (2.0 equiv.), dichloromethane (20 mL) and water (10 mL) is added to round bottom flask (50 mL) equipped with a magnetic stir bar. The flask is then connected to a reflux condenser and placed in an oil bath. The mixture is stirred for 3 hours at 50° C. Then, the mixture is filtered, and the solvent is removed via rotary evaporator. The residue is then extracted with dichloromethane (2×30 mL). The combined organic fractions are extracted with water to remove sodium halide salts, dried over Na₂SO₄ and reconcentrated under vacuum to give the desired product bis(diphenyltinstyrenecarboxylate)propane as a white solid.

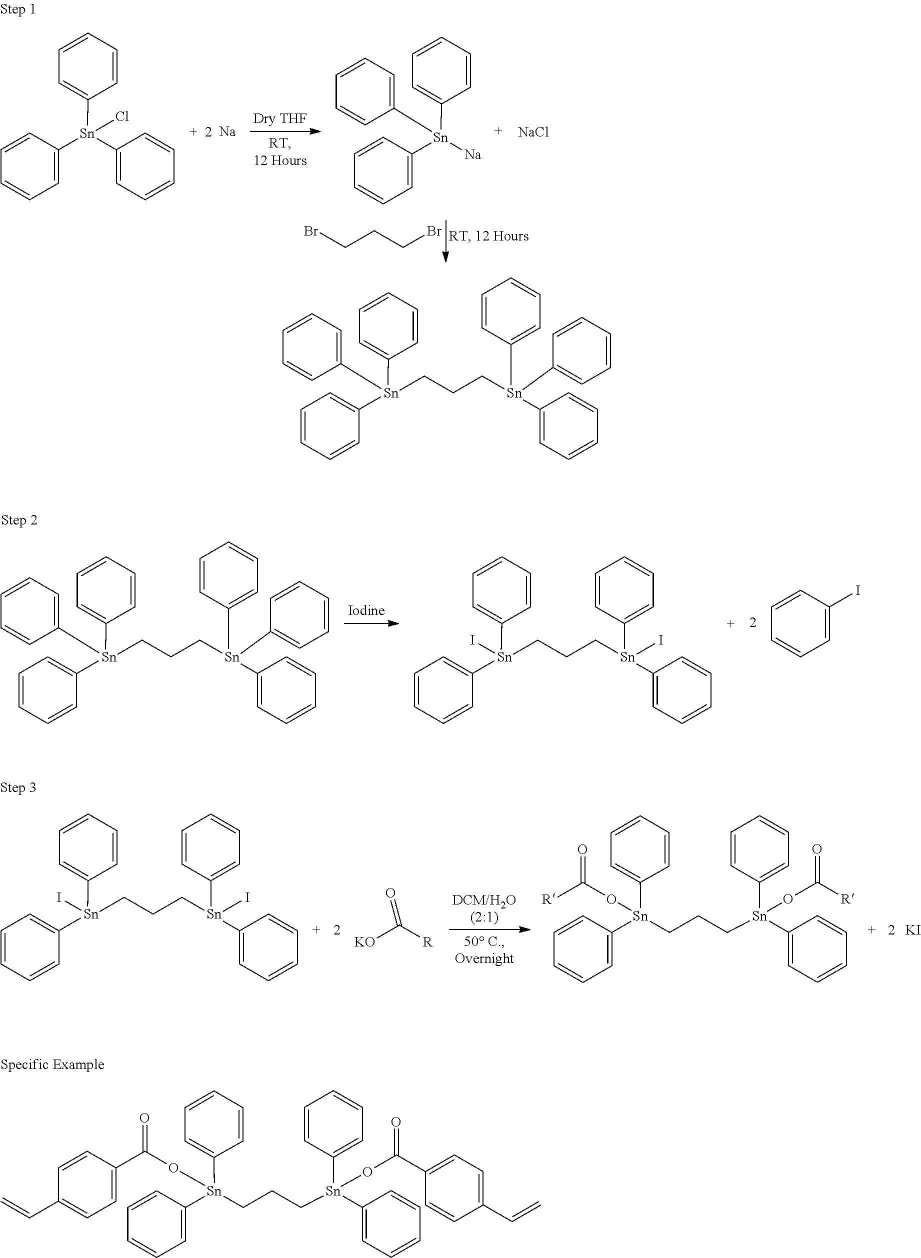

### Type 8: Bridging Metal Complexes Synthesis.

Type 8: Oxalate Bridge Metal Complexes: Metal precursor (R_{f/2}MX₂, 2.0 equiv.) and silver oxalate (1.0 equiv.) were added to a round bottom flask (100 mL) with a magnetic stir bar followed by dichloromethane (50 mL). The flask was sealed with a septa, the mixture was stirred for 24 hours at room temperature. Then, the mixture was filtered, and the volume of the mother-liquor was reduced to approximately 20 mL via rotary evaporator, and a white powder started to precipitate out of solution. The compound was purified by crystallization from hot methanol.

Type 8: Sulfate Bridge Metal Complexes: Metal precursor (R_{f/2}MX₂, 2.0 equiv.) and silver sulfate (1.0 equiv.) were added to a round bottom flask (100 mL) with a magnetic stir bar followed by methanol (50 mL). The flask was sealed with a septa, the mixture was stirred for 24 hours at room temperature. Then, the mixture was filtered, and the volume of the mother-liquor was reduced to approximately 20 mL via rotary evaporator, and a white powder started to precipitate out of solution. The compound was purified by crystallization from hot methanol.

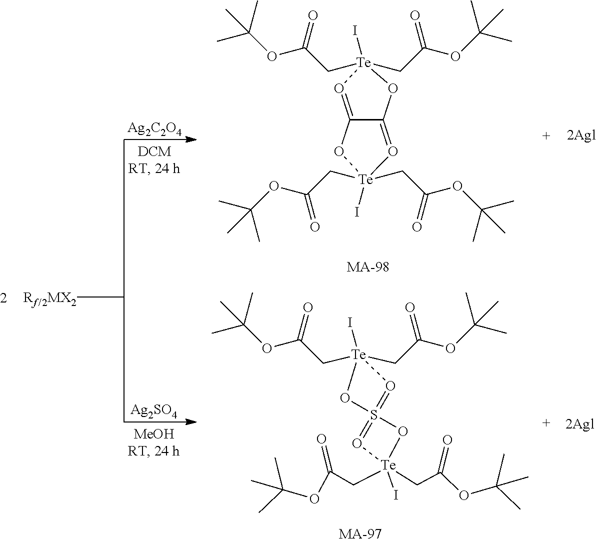

Bridging Metal Complexes Synthesis; M=Te, X=I, R=C(CH₃)₃O₂CCH₂, C₂H₅OCOCO, C₂H₅OCOCOCH₂, C₂H₅OCOCO, ClC₄H₅CO are MA-98, MA-146, MA-165, MA-246, and R=C6H5OCH3, X=Cl is MA-145, and M=Te, X=OMe, R=C(CH₃)₃O₂CCH₂, C₂H₃C₆H₄CH₂ are MA-150, MA-151 and M=Sb, X=OMe, R=C(CH₃)═CH₂, C₆H₄CH═CH₂ are RB-5, and RB-12, respectively.

### Type 9: Telluric Acid Clusters. (Prophetic)

Preparation of Tellurium Acid Clusters: The reaction is carried out under a nitrogen atmosphere with the use of standard Schlenk techniques. The Telluric acid (1.0 equiv.) is added to the solution of methanol (30 mL) together with potassium hydroxide (4.0 equiv.), and the mixture stir for 0.5 h. After the addition of metal salt (2.0 equiv.) the mixture is stirred at 50° C. for 12 h and then filtered. The solvent is gradually removed by evaporation under vacuum until a solid product is obtained. The solid is then recrystallized from diethyl ether, and colorless crystals of complex are collected.¹

### Telluric Acid Cluster Synthesis:

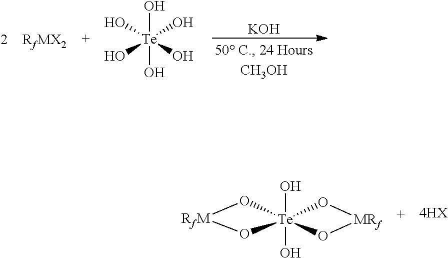

### Where

X=I, Cl
M=Te, Sb, Sn

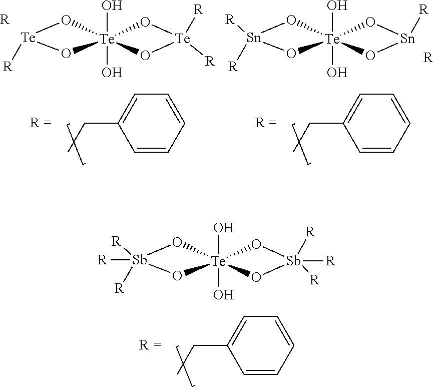

A composition from Table 1 below is formulated into a lithographic composition and is deposited atop a substrate. Table I includes, for each horizontal line, a composition of the formula [MₐN_{b}O_{c}H_{d}BₑR_{f}X_{g}(WCOR′)ₕ(OR″)ᵢ(WCOCOY)ⱼ(COY)ₖ]^{l}. In embodiments, each composition may have (I) equal to a charge on an ion or a complex selected from: −4, −3, −2, −1, 0, +1, +2, +3, +4.

TABLE I

| | Compound | | | | | | | | | | | | | |
|---|---|---|---|---|---|---|---|---|---|---|---|---|---|---|
| # | Number | M | a | N | b | c | d | B | e | R | f | X | g | WCOR′ |
| 1 | MA-192 | 1 | 1 | Te | 2 | 6 | 1 | 0 | 0 | p-C6H4OCH3 | 4 | 0 | 0 | |
| 2 | MA-218 | 1 | 1 | Te | 2 | 6 | 1 | 0 | 0 | 0 | 0 | 0 | 0 | |
| 3 | MA-227 | 1 | 1 | Te | 2 | 6 | 1 | 0 | 0 | 0 | 0 | 0 | 0 | |
| 4 | MA-229 | 1 | 1 | Te | 2 | 6 | 1 | 0 | 0 | 0 | 0 | 0 | 0 | 0 |
| 5 | MA-251 | 1 | 1 | Sn | 2 | 6 | 1 | 0 | 0 | CH2C6H5 | 4 | 0 | 0 | 0 |
| 6 | RB-104 | Sb | 2 | 0 | 0 | 1 | 0 | 0 | 0 | C6H5 | 6 | 0 | 0 | O2CCH2Cl |
| 7 | RB-109 | Sb | 2 | 0 | 0 | 1 | 0 | 0 | 0 | CH(CH3)2 | 6 | 0 | 0 | O2CCH2CH2Cl |
| 8 | RB-110 | Sb | 2 | 0 | 0 | 1 | 0 | 0 | 0 | CH(CH3)2 | 6 | 0 | 0 | O2CC6H4CH═CH2 |
| 9 | RB-54 | Sb | 2 | 0 | 0 | 1 | 0 | 0 | 0 | C6H5 | 6 | 0 | 0 | O2CCH2CH2Cl |
| 10 | RB-124 | Sb | 2 | 0 | 0 | 1 | 0 | 0 | 0 | C6H4CH═CH2 | 6 | 0 | 0 | O2CCH2CH2Cl |
| 11 | MA-256 | Te | 1 | Sn | 6 | 6 | 0 | 0 | 0 | CH2C6H5 | 6 | 0 | 0 | 0 |
| 12 | NU-158 | Te | 1 | Sb | 3 | 6 | 0 | 0 | 0 | C6H11 | 9 | 0 | 0 | 0 |
| 13 | NU-160 | Te | 1 | Sb | 3 | 6 | 0 | 0 | 0 | C6H4CH2CH3 | 9 | 0 | 0 | 0 |
| 14 | NU-82 | Te | 1 | Sb | 3 | 6 | 0 | 0 | 0 | C(CH3)═CH2 | 9 | 0 | 0 | 0 |
| 15 | NU-87 | Te | 1 | Sb | 3 | 6 | 0 | 0 | 0 | C6H4CH═CH2 | 9 | 0 | 0 | 0 |
| 16 | MA-129 | Te | 4 | 0 | 0 | 6 | 0 | 0 | 0 | p-C6H4OCH3 | 6 | 0 | 0 | |
| 17 | MA-176 | Te | 4 | 0 | 0 | 6 | 0 | 0 | 0 | CH2C6H5 | 6 | 0 | 0 | 0 |
| 18 | MA-207 | Te | 4 | 0 | 0 | 6 | 0 | 0 | 0 | C6H4CH═CH2 | 3 | 0 | 0 | CH2CO2C(CH3)3 |
| 19 | MA-216 | Te | 4 | 0 | 0 | 6 | 0 | 0 | 0 | 0 | 0 | 0 | 0 | |
| 20 | RB-129 | Sn | 2 | 0 | 0 | 0 | 0 | CH2 | 1 | C6H5 | 4 | 0 | 0 | O2CC6H4CH═CH2 |
| 21 | RB-56 | Sb | 2 | 0 | 0 | 0 | 0 | CH2 | 1 | C6H5 | 4 | 0 | 0 | O2CCH2CH2Cl |
| 22 | MA-145 | Te | 2 | 0 | 0 | 0 | 0 | C2O4 | 1 | p-C6H4OCH3 | 4 | Cl | 2 | |
| 23 | MA-146 | Te | 2 | 1 | 2 | 0 | 0 | C2O4 | 1 | 0 | 0 | 0 | 0 | 0 |
| 24 | MA-150 | Te | 2 | 0 | 0 | 0 | 0 | C2O4 | 1 | 0 | 0 | 0 | 0 | CH2CO2C(CH3)3 |
| 25 | MA-151 | Te | 2 | 0 | 0 | 0 | 0 | C2O4 | 1 | CH2C6H4CH═CH2 | 4 | 0 | 0 | 0 |
| 26 | MA-165 | Te | 2 | 1 | 2 | 0 | 0 | C2O4 | 1 | 0 | 0 | 0 | 0 | 0 |
| 27 | MA-166 | Te | 2 | 1 | 2 | 0 | 0 | C2O4 | 1 | 0 | 0 | 0 | 0 | 0 |
| 28 | MA-246 | Te | 2 | 1 | 2 | 0 | 0 | C2O4 | 1 | 0 | 0 | 0 | 0 | 0 |
| 29 | MA-97 | Te | 2 | 1 | 2 | 0 | 0 | SO4 | 1 | 0 | 0 | 0 | 0 | CH2CO2C(CH3)3 |
| 30 | MA-98 | Te | 2 | 1 | 2 | 0 | 0 | C2O4 | 1 | 0 | 0 | 0 | 0 | CH2CO2C(CH3)3 |
| 31 | RB-12 | Sb | 2 | 0 | 0 | 0 | 0 | C2O4 | 1 | C6H4CH═CH2 | 6 | 0 | 0 | 0 |
| 32 | RB-5 | Sb | 2 | 0 | 0 | 0 | 0 | C2O4 | 1 | C(CH3)═CH2 | 6 | 0 | 0 | 0 |
| 33 | MA-155 | Te | 2 | 1 | 0 | 0 | 0 | PO4 | 1 | 0 | 0 | 0 | 0 | CH2CO2C(CH3)3 |
| 34 | MA-233 | Te | 4 | 0 | 0 | 0 | 0 | 0 | 0 | C6H5 | 4 | 0 | 0 | O2CC6H4CH═CH2 |
| 35 | MA-93 | Te | 2 | 0 | 0 | 2 | 0 | 0 | 0 | 0 | 0 | | | CH2CO2C(CH3)3 |
| 36 | NU-119 | Te | 2 | 0 | 0 | 0 | 0 | (CH₂)₂C₆H₄(CH₂)₂ | 1 | 0 | 0 | 0 | 0 | O2CC(CH3)═CH2 |
| 37 | NU-139 | Te | 2 | 0 | 0 | 0 | 0 | (CH₂)₂C₆H₄(CH₂)₂ | 1 | 0 | 0 | 0 | 0 | O2CCH═CH2 |
| 38 | NU-141 | Te | 2 | 0 | 0 | 0 | 0 | (CH₂)₂C₆H₄(CH₂)₂ | 1 | 0 | 0 | 0 | 0 | O2CC(CH3)3 |
| 39 | NU-143 | Te | 4 | 0 | 0 | 6 | 0 | (CH₂)₂C₆H₄(CH₂)₂ | 1 | 0 | 0 | 0 | 0 | 0 |
| 40 | NU-176 | Te | 1 | Sn | 4 | 6 | 2 | 0 | 0 | C6H5 | 12 | 0 | 0 | 0 |
| 41 | RB-55 | Sb | 2 | Te | 1 | 0 | 0 | 0 | 0 | C6H5 | 4 | 0 | 0 | 0 |
| 42 | RB-60 | Te | 1 | Sb | 1 | 0 | 0 | 0 | 0 | C6H5/p-C6H4OCH3 | 1/2 | 0 | 0 | |

| # | h | OR″ | i | WCOCOY | j | COY | k | l | Comment | Type |
|---|---|---|---|---|---|---|---|---|---|---|
| 1 | | 0 | 0 | 0 | 0 | 0 | 0 | 0 | New | 1 |
| 2 | | 0 | 0 | 0 | 0 | COC6H5 | 4 | 0 | New | 1 |
| 3 | | 0 | 0 | 0 | 0 | COC6H4OCH3 | 4 | 0 | New | 1 |
| 4 | 0 | 0 | 0 | COCO2C(CH3)3 | 4 | 0 | 0 | 0 | New | 1 |
| 5 | 0 | 0 | 0 | 0 | 0 | 0 | 0 | 0 | New | 1 |
| 6 | 2 | 0 | 0 | 0 | 0 | 0 | 0 | 0 | New | 2 |
| 7 | 2 | 0 | 0 | 0 | 0 | 0 | 0 | 0 | New | 2 |
| 8 | 2 | 0 | 0 | 0 | 0 | 0 | 0 | 0 | New | 2 |
| 9 | 2 | 0 | 0 | 0 | 0 | 0 | 0 | 0 | New | 2 |
| 10 | 2 | 0 | 0 | 0 | 0 | 0 | 0 | 0 | New | 2 |
| 11 | 0 | 0 | 0 | 0 | 0 | 0 | 0 | 0 | New | 4 |
| 12 | 0 | 0 | 0 | 0 | 0 | 0 | 0 | 0 | New | 4 |
| 13 | 0 | 0 | 0 | 0 | 0 | 0 | 0 | 0 | New | 4 |
| 14 | 0 | 0 | 0 | 0 | 0 | 0 | 0 | 0 | New | 4 |
| 15 | 0 | 0 | 0 | 0 | 0 | 0 | 0 | 0 | New | 4 |
| 16 | | 0 | 0 | 0 | 0 | 0 | 0 | 0 | New | 5 |
| 17 | 0 | 0 | 0 | 0 | 0 | 0 | 0 | 0 | New | 5 |
| 18 | 3 | 0 | 0 | 0 | 0 | 0 | 0 | 0 | New | 5 |
| 19 | | 0 | 0 | | | C(O)C6H5 | 6 | 0 | New | 5 |
| 20 | 2 | 0 | 0 | 0 | 0 | 0 | 0 | 0 | New | 6 |
| 21 | 4 | 0 | 0 | 0 | 0 | 0 | 0 | 0 | New | 6 |
| 22 | | 0 | 0 | 0 | 0 | 0 | 0 | 0 | New | 8 |
| 23 | 0 | 0 | 0 | COCO2CH2CH3 | 4 | 0 | 0 | 0 | New | 8 |
| 24 | 4 | OCH3 | 2 | 0 | 0 | 0 | 0 | 0 | New | 8 |
| 25 | 0 | OCH3 | 2 | 0 | 0 | 0 | 0 | 0 | New | 8 |
| 26 | 0 | 0 | 0 | CH2COCO2CH2CH3 | 4 | 0 | 0 | 0 | New | 8 |
| 27 | 0 | 0 | 0 | COCO2CH2CH3 | 4 | 0 | 0 | 0 | New | 8 |
| 28 | 0 | 0 | 0 | 0 | 0 | COC6H4Cl | 4 | 0 | New | 8 |
| 29 | 4 | 0 | 0 | 0 | 0 | 0 | 0 | 0 | New | 8 |
| 30 | 4 | 0 | 0 | 0 | 0 | 0 | 0 | 0 | New | 8 |
| 31 | 0 | OCH3 | 2 | 0 | 0 | 0 | 0 | 0 | New | 8 |
| 32 | 0 | OCH3 | 2 | 0 | 0 | 0 | 0 | 0 | New | 8 |
| 33 | 4 | 0 | 0 | 0 | 0 | 0 | 0 | 0 | New | |
| 34 | 4 | 0 | 0 | 0 | 0 | 0 | 0 | 0 | New | |
| 35 | 4 | 0 | 0 | 0 | 0 | 0 | 0 | 0 | New | |
| 36 | 4 | 0 | 0 | 0 | 0 | 0 | 0 | 0 | New | |
| 37 | 4 | 0 | 0 | 0 | 0 | 0 | 0 | 0 | New | |
| 38 | 4 | 0 | 0 | 0 | 0 | 0 | 0 | 0 | New | |
| 39 | 0 | 0 | 0 | 0 | 0 | 0 | 0 | 0 | New | |
| 40 | 0 | 0 | 0 | 0 | 0 | 0 | 0 | 0 | New | |
| 41 | 0 | 0 | 0 | 0 | 0 | 0 | 0 | 0 | New | |
| 42 | | 0 | 0 | 0 | 0 | 0 | 0 | 0 | New | |

The entire disclosure of all applications, patents, and publications cited herein are herein incorporate by reference in their entirety. While the foregoing is directed to embodiments of the present disclosure, other and further embodiments of the disclosure may be devised without departing from the basic scope thereof.

The present invention is not to be limited by the above description, but to be defined by the appended claims and their equivalents.

What is claimed is:

1. A lithographic composition represented by the below formula,
[MₐN_{b}O_{c}H_{d}BₑR_{f}X_{g}(WCOR′)ₕ(OR″)ᵢ(WCOCOY)ⱼ(COY)ₖ]^{l},
said composition comprising:
R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH₂C₆H₄F, —CH₂C₆H₃F₂, —CH₂C₆H₂F₃, —CH₂C₆F₅, —CH(CH₃)C₆H₅, —CH(CH₃)C₁₀H₇, —CH(CH₃)C₆H₄C₆H₅, or —CH(CH₃)C₆H₄C(CH₃)₃;
R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, or —C₆H₄Cl;
R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH₂CH═CH₂, —CH₂C≡CH, —CH₂C≡N, —CH₂C₆H₅, —C₆H₄CH═CH₂, —CH(CH₃)C₆H₅, or —C₆H₄C(CH₃)=CH₂;
M is each independently an element selected from the group of antimony (Sb), tellurium (Te), tin (Sn) or iodine (I);
N, when present, is each independently an element selected from the group consisting of tin (Sn), antimony (Sb), tellurium (Te), bismuth (Bi) iodine (I) and indium (In);
H, when present, is each independently hydrogen;
O is each independently oxygen;
B, when present, is each independently selected from W, C₂O₄, SO₄, PO₄, (CH₂)₂C₆H₄(CH₂)₂, —OO—, —OCH₂O—, —OCH₂CH₂O—, —OCH₂CH₂CH₂O—, —OCH₂CH₂CH₂CH₂O—, —OC₆H₄O—, —OCH₂C₆H₄CH₂O—, —OCH₂CH═CHCH₂O—, —OCH₂C≡CCH₂O—, —SCH₂CH₂S—, —SCH₂CH₂CH₂S—, —SCH₂CH₂CH₂CH₂S—, —SC₆H₄S—, —SCH₂C₆H₄CH₂S—, —SCH₂CH═CHCH₂S—, —SCH₂C≡CCH₂S—, —O₂CCH₂CO₂—, —O₂CCH₂CH₂CO₂—, —O₂CCH₂C₆H₄CH₂CO₂—, —NHC═ONH—, —CH₂—, —CH₂CH₂—, —CH₂CH₂CH₂, —CH(CH₃)—, —CH₂CH═CHCH₂—, —CH₂C≡CCH₂—, or —CH₂C₆H₄CH₂—;
X, when present, is each independently selected from F, Cl, Br, CN, or C₂O₄;
W, when present, is each independently selected from the group consisting of —CH₂, NR′, S, and O; and
Y when present, is each independently selected from the group consisting of —WR′, and —R′, wherein
a=1-8; b=0-5; c=0-20; d=0-20; e=0-10; f=0-20; g=0-20; h=0-5; i=0-5; j=0-5; k=0-6; and l=charge on an ion or a complex selected from: −4, −3, −2, −1, 0, +1, +2, +3, +4.

2. The lithographic composition of claim 1, wherein M=I, a=1, N=Sn, Te, Sb, b=2, c=6, d=1, B=0, e=0, R=R; f=0 or 4 when N=Sn or Te, and f=0 or 6 when N=Sb; X=0, g=0, WCOR′=0, h=0, R″=0, i=0, WCOCOY=COCO₂C(CH₃)₃, j=0 or 4 when N=Sn or Te, and j=0 or 6 when N=Sb; COY=COC₆H₅ or COC₆H₄OCH₃, k=0 or 4 when N=Sn or Te, and k=0 or 6 when N=Sb; l=0.

3. The lithographic composition of claim 2, wherein the composition is represented by the below formula:

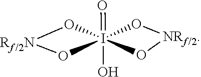

4. The lithographic composition of claim 3, wherein M=I, a=1, N=Sn, b=2, c=6, d=1, B=0, e=0, X=0, g=0, WCOR′=0, h=0, R″=0, i=0, WCOCOY=0, j=0, COY=0, k=0, l=0;
R is independently an aromatic or aliphatic hydrocarbon selected from: —C₆H₅, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH(CH₃)C₆H₅, —CH(CH₃)C₁₀H₇, or —CH(CH₃)C₆H₄C(CH₃)₃;
and f=4.

5. The lithographic composition of claim 3, wherein M=I, a=1, N=Te, b=2, c=6, d=1, B=0, e=0, X=0, g=0, WCOR′=0, h=0, R″=0, i=0, WCOCOY=0, j=0, COY=0, k=0, l=0; R is independently an aromatic or aliphatic hydrocarbon selected from: —C₆H₅, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH(CH₃)C₆H₅, —CH(CH₃)C₁₀H₇, or —CH(CH₃)C₆H₄C(CH₃)₃; and f=4.

6. The lithographic composition of claim 3, wherein M=I, a=1, N=Sb, b=2, c=6, d=1, B=0, e=0, X=0, g=0, WCOR′=0, h=0, R″=0, i=0, WCOCOY=0, j=0, COY=0, k=0, l=0; R is independently an aromatic or aliphatic hydrocarbon selected from: —C₆H₅, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH(CH₃)C₆H₅, —CH(CH₃)C₁₀H₇, or —CH(CH₃)C₆H₄C(CH₃)₃; and f=6.

7. The lithographic composition of claim 1, wherein M=Sb, Sn, Te; a=2, N=0, b=0, c=1, d=0, B=0, e=0, R=R; f=4 when N=Sn or Te, and f=6 when N=Sb; X=0, g=0, W=—O—, R′=R′, h=2, R″=0, i=0, WCOCOY=0, j=0, COY=0, k=0, 1=0.

8. The lithographic composition of claim 7, wherein the composition is represented by the below formula:

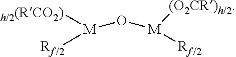

9. The lithographic composition of claim 8, wherein: R is independently an aromatic or aliphatic hydrocarbon selected from: —C₆H₅, —CH(CH₃)₂, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH(C₆H₅)₂, or —CH₂C₆H₄C(CH₃)₃; R′ is independently an aromatic or aliphatic hydrocarbon selected from: —H, —C(CH₃)₃, —C₆H₅, —CH═CH₂, —C(CH₃)=CH₂, —CH₂C₆H₅, —C₆H₄CH═CH₂, —C₆H₄C(CH₃)=CH₂, —CH₂CH₂Cl, —CH₂CH₂Br, —CH₂CH₂I, —CH₂CH₂OCH₃, —CH₂CH₂CN, —CHCl(CH₃), —CHBr(CH₃), —CHI(CH₃), —CHOCH₃(CH₃), —CHCN(CH₃), —CH₂Cl, —CH₂Br, —CH₂I, —CH₂OCH₃, or —CH₂CN.

10. The lithographic composition of claim 1, wherein M=Sb, Sn, Te; a=2, N=0, b=0, c=0, d=0, e=1, R=R; f=4 when N=Sn or Te, and f=6 when N=Sb; X=0, g=0, W=—O—, R′=R′, h=2, R″=0, i=0, WCOCOY=0, j=0, COY=0, k=0, l=0 and B=—OO—, —OCH₂O—, —OCH₂CH₂O—, —OCH₂CH₂CH₂O—, —OCH₂CH₂CH₂CH₂O—, —OC₆H₄O—, —OCH₂C₆H₄CH₂O—, —OCH₂CH═CHCH₂O—, —OCH₂C≡CCH₂O—, —SCH₂CH₂S—, —SCH₂CH₂CH₂S—, —SCH₂CH₂CH₂CH₂S—, —SC₆H₄S—, —SCH₂C₆H₄CH₂S—, —SCH₂CH═CHCH₂S—, —SCH₂C≡CCH₂S—, —O₂CCH₂CO₂—, —O₂CCH₂CH₂CO₂—, or —NHC═ONH—.

11. The lithographic composition of claim 10, wherein the composition is represented by the below formula:

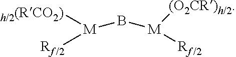

12. The lithographic composition of claim 11, wherein: R is independently an aromatic or aliphatic hydrocarbon selected from: —C₆H₅, —CH(CH₃)₂, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH(C₆H₅)₂, or —CH₂C₆H₄C(CH₃)₃; B is each independently selected from —OO—, —OCH₂CH₂O—, —OCH₂CH₂CH₂O—, —OC₆H₄O—, —OCH₂C₆H₄CH₂O—, —OCH₂CH═CHCH₂O—, —OCH₂C≡CCH₂O—, —SCH₂CH₂S—, —SCH₂C₆H₄CH₂S—, —SCH₂CH═CHCH₂S—, —SCH₂C≡CCH₂S—, —O₂CCH₂CO₂—, —O₂CCH₂CH₂CO₂—, —O₂CCH₂C₆H₄CH₂CO₂—; W is —O—; R′ is independently an aromatic or aliphatic hydrocarbon selected from: —H, —C(CH₃)₃, —C₆H₅, —CH═CH₂, —C(CH₃)=CH₂, —CH₂C₆H₅, —C₆H₄CH═CH₂, —C₆H₄C(CH₃)=CH₂, —CH₂CH₂Cl, —CH₂CH₂Br, —CH₂CH₂I, —CH₂CH₂OCH₃, —CH₂CH₂CN, —CHCl(CH₃), —CHBr(CH₃), —CHI(CH₃), —CHOCH₃(CH₃), —CHCN(CH₃), —CH₂Cl, —CH₂Br, —CH₂I, —CH₂OCH₃, or —CH₂CN.

13. The lithographic composition of claim 1, wherein M=Te, a=1, N=Sn or Sb, b=3, c=6, d=0, B=0, e=0, R=R; f=6 when N=Sn, and f=9 when N=Sb; X=0, g=0, WCOR′=0, h=0, R″=0, i=0, WCOCOY=0, j=0, COY=0, k=0, l=0.

14. The lithographic composition of claim 13, wherein the composition is represented by the below formula:

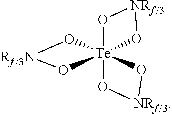

15. The lithographic composition of claim 14, wherein: R is independently an aromatic or aliphatic hydrocarbon selected from: —C₆H₅, —CH(CH₃)₂, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH(C₆H₅)₂, or —CH₂C₆H₄C(CH₃)₃.

16. The lithographic composition of claim 1, wherein M=Te, a=4, N=0, b=0, c=6, d=0, B=0, e=0, R=R, f=0 or 6, X=0, g=0, WCOR′=0, h=0, R″=0, i=0, WCOCOY=0, j=0, l=0, COY=COC₆H₅, k=0 or 6.

17. The lithographic composition of claim 16, wherein M=Te, a=4, N=0, b=0, c=6, d=0, B=0, e=0, X=0, g=0, WCOR′=0, h=0, R″=0, i=0, WCOCOY=0, j=0, COY=0, k=0, l=0,
R is independently an aromatic or aliphatic hydrocarbon selected from: —C₆H₅, —CH(CH₃)₂, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH(C₆H₅)₂, or —CH₂C₆H₄C(CH₃)₃; f=6.

18. The lithographic composition of claim 17, wherein the composition is represented by the below formula:

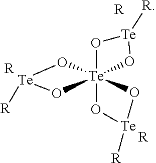

19. The lithographic composition of claim 1, wherein M=Sb or Sn, a=2, N=0, b=0, c=0, d=0, B=CH₂, e=1, R=R, f=4, X=0, g=0, W=—O—, R′=R′; h=2 if M=Sn, or h=4 if M=Sb; R″=0, i=0, WCOCOY=0, j=0, COY=0, k=0, l=0.

20. The lithographic composition of claim 19, wherein the composition is represented by the below formula:

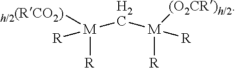

21. The lithographic composition of claim 20, wherein: R is independently an aromatic or aliphatic hydrocarbon selected from: —C₆H₅, —CH(CH₃)₂, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH(C₆H₅)₂, or —CH₂C₆H₄C(CH₃)₃; R′ is independently an aromatic or aliphatic hydrocarbon selected from: —H, —C(CH₃)₃, —C₆H₅, —CH═CH₂, —C(CH₃)=CH₂, —CH₂C₆H₅, —C₆H₄CH═CH₂, —C₆H₄C(CH₃)=CH₂, —CH₂CH₂Cl, —CH₂CH₂Br, —CH₂CH₂I, —CH₂CH₂OCH₃, —CH₂CH₂CN, —CHCl(CH₃), —CHBr(CH₃), —CHI(CH₃), —CHOCH₃(CH₃), —CHCN(CH₃), —CH₂Cl, —CH₂Br, —CH₂I, —CH₂OCH₃, or —CH₂CN.

22. The lithographic composition of claim 1, wherein M=Sb or Sn, a=2, N=0, b=0, c=0, d=0; B=—CH₂CH₂—, —CH₂CH₂CH₂—, —CH(CH₃)—, —CH₂CH═CHCH₂—, —CH₂C≡CCH₂—, or —CH₂C₆H₄CH₂—; e=1, R=R, f=4, X=0, g=0, W=—O—, R′=R′; h=2 if M=Sn, or h=4 if M=Sb; R″=0, i=0, WCOCOY=0, j=0, COY=0, k=0, l=0.

23. The lithographic composition of claim 22, wherein the composition is represented by the below formula:

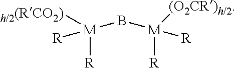

24. The lithographic composition of claim 23, wherein, R is independently an aromatic or aliphatic hydrocarbon selected from: —C₆H₅, —CH(CH₃)₂, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH(C₆H₅)₂, or —CH₂C₆H₄C(CH₃)₃; B is each independently selected from —CH₂—, —CH₂CH₂—, —CH₂CH₂CH₂—, —CH(CH₃)—, —CH₂CH═CHCH₂—, —CH₂C≡CCH₂—, or —CH₂C₆H₄CH₂—; W is —O—; R′ is independently an aromatic or aliphatic hydrocarbon selected from: —H, —C(CH₃)₃, —C₆H₅, —CH═CH₂, —C(CH₃)=CH₂, —CH₂C₆H₅, —C₆H₄CH═CH₂, —C₆H₄C(CH₃)=CH₂, —CH₂CH₂Cl, —CH₂CH₂Br, —CH₂CH₂I, —CH₂CH₂OCH₃, —CH₂CH₂CN, —CHCl(CH₃), —CHBr(CH₃), —CHI(CH₃), —CHOCH₃(CH₃), —CHCN(CH₃), —CH₂Cl, —CH₂Br, —CH₂I, —CH₂OCH₃, or —CH₂CN.

25. The lithographic composition of claim 1, wherein M=Sb, Te or Sn, a=2, N=0, b=0, c=0, d=0, B=—O₂CCO₂— or —O₂SO₂—, e=1, R=R, f=0 or 4 if M=Te or Sn, f=0 or 6 if M=Sb;
X=X, g=0 or 2; WCOR′=CH₂CO₂C(CH₃)₃, h=0 or 4 if M=Te or Sn, h=0 or 6 if M=Sb;
OR″=OCH₃ or OC(CH₃)₃, i=0 or 2, WCOCOY=CH₂COCO₂CH₂CH₃ or COCO₂CH₂CH₃, j=0 or 4 if M=Te or Sn, j=0 or 6 if M=Sb; COY=COC₆H₄Cl, k=0 or 4 if M=Te or Sn, k=0 or 6 if M=Sb; l=0.

26. The lithographic composition of claim 25, wherein M=Sb, Te or Sn, a=2, N=0, b=0, c=0, d=0, B=—O₂CCO₂— or —O₂SO₂—, e=1, X=X, g=0 or 2, WCOR′=0, h=0, R″=R″ i=0 or 2, WCOCOY=0, j=0, COY=0, k=0, l=0; R is independently an aromatic or aliphatic hydrocarbon selected from: —C₆H₅, —CH(CH₃)₂, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH(C₆H₅)₂, or —CH₂C₆H₄C(CH₃)₃; f=4 if M=Te or Sn, f=6 if M=Sb; B is each independently selected from —O₂CCO₂— or —O₂SO₂—.

27. The lithographic composition of claim 26, wherein the composition is represented by the below formula:

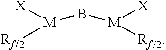

28. The lithographic composition of claim 1, wherein M=Te, a=1, N=Sn, Te or Sb, b=2, c=6, d=2, B=0, e=0, R=R; f=4 if N=Sn or Te or f=6 if N=Sb; X=O, g=0, WCOR′=0, h=0, R″=0, i=0, WCOCOY=0, j=0, COY=0, k=0, l=0.

29. The lithographic composition of claim 28, wherein the composition is represented by the below formula:

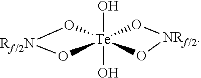

30. The lithographic composition of claim 29, wherein: R is independently an aromatic or aliphatic hydrocarbon selected from: —C₆H₅, —CH(CH₃)₂, —C(CH₃)₃, —C(CH₃)=CH₂, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —CH₂C₆H₄OCH₃, —C₆H₁₁, —CH₂C₁₀H₇, —CH(C₆H₅)₂, or —CH₂C₆H₄C(CH₃)₃.

31. The lithographic composition of claim 1, wherein the formula comprises a multinuclear cluster structure selected from the group consisting of: 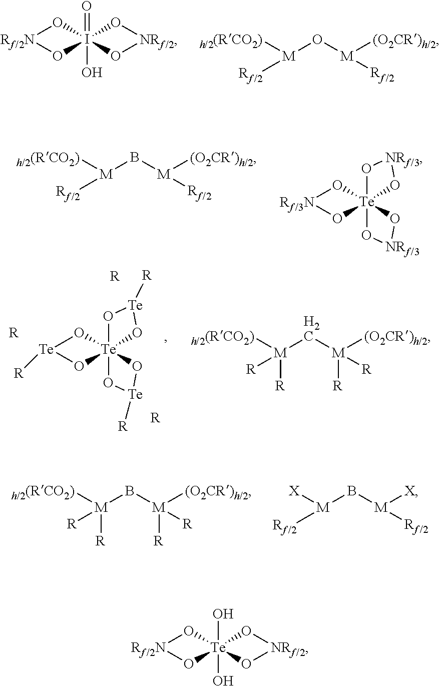
and mixtures there of.

32. A coating solution comprising: an organic solvent, and an organometallic composition represented by the formula
[MₐN_{b}O_{c}H_{d}BₑR_{f}X_{g}(WCOR′)ₕ(OR″)ᵢ(WCOCOY)ⱼ(COY)ₖ]^{l},
wherein:
R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —CH₂C₁₀H₇, —CH₂C₆H₄C₆H₅, —CH(C₆H₅)₂, —CH₂C₆H₄C(CH₃)₃, —CH₂C₆H₄F, —CH₂C₆H₃F₂, —CH₂C₆H₂F₃, —CH₂C₆F₅, —CH(CH₃)C₆H₅, —CH(CH₃)C₁₀H₇, —CH(CH₃)C₆H₄C₆H₅, or —CH(CH₃)C₆H₄C(CH₃)₃;
R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)=CH₂, —CH₂CH═CH₂, —CH₂C≡CH, —CH₂C≡N, —CH₂C₆H₅, —C₆H₄CH═CH₂, —C₆H₄C(CH₃)=CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, or —C₆H₄Cl;
R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH₂CH═CH₂, —CH₂C≡CH, —CH₂C≡N, —CH₂C₆H₅, —C₆H₄CH═CH₂, —CH(CH₃)C₆H₅, or —C₆H₄C(CH₃)=CH₂;
M is each independently an element selected from the group of antimony (Sb), tellurium (Te), tin (Sn) or iodine (I);
N, when present, is each independently an element selected from the group consisting of tin (Sn), antimony (Sb), tellurium (Te), bismuth (Bi) iodine (I) and indium (In);
H, when present, is each independently hydrogen;
O is each independently oxygen;
B, when present, is each independently selected from W, C₂O₄, SO₄, PO₄, (CH₂)₂C₆H₄(CH₂)₂, —OO—, —OCH₂O—, —OCH₂CH₂O—, —OCH₂CH₂CH₂O—, —OCH₂CH₂CH₂CH₂O—, —OC₆H₄O—, —OCH₂C₆H₄CH₂O—, —OCH₂CH═CHCH₂O—, —OCH₂C≡CCH₂O—, —SCH₂CH₂S—, —SCH₂CH₂CH₂S—, —SCH₂CH₂CH₂CH₂S—, —SC₆H₄S—, —SCH₂C₆H₄CH₂S—, —SCH₂CH═CHCH₂S—, —SCH₂C≡CCH₂S—, —O₂CCH₂CO₂—, —O₂CCH₂CH₂CO₂—, —O₂CCH₂C₆H₄CH₂CO₂—, —NHC═ONH—, —CH₂—, —CH₂CH₂—, —CH₂CH₂CH₂, —CH(CH₃)—, —CH₂CH═CHCH₂—, —CH₂C≡CCH₂—, or —CH₂C₆H₄CH₂—;
X, when present, is each independently selected from F, Cl, Br, CN, or C₂O₄;
W, when present, is each independently selected from the group consisting of —CH₂, NR′, S, and O; and
Y when present, is each independently selected from the group consisting of —WR′, and —R′, wherein
a=1-8; b=0-5; c=0-20; d=0-20; e=0-10; f=0-20; g=0-20; h=0-5; i=0-5; j=0-5; k=0-6; and l=charge on an ion or a complex selected from: −4, −3, −2, −1, 0, +1, +2, +3, +4, or a mixture thereof.

33. The coating solution of claim 32, wherein the solvent is an alcohol, an ester, or a mixture thereof.

34. A method for forming a radiation patternable coating, the method comprising: contacting a coating solution of claim 33 with a substrate under conditions suitable for forming a film atop the substrate.

35. The method of claim 34, wherein the substrate is heated to a temperature from about 30 degrees Celsius to about 250 degrees Celsius for about 0.5 minutes to about 30 minutes.

36. The method of claim 34, wherein the coating solution is spin coated to form a film atop the substrate.
